(19) United States
(12) Patent Application Publication
(10) Pub. No.: US 2022/0119350 A1
(43) Pub. Date: Apr. 21, 2022

(54) COMPOUNDS FOR THE TREATMENT OF OCULAR DISEASE

(71) Applicant: GLAUKOS CORPORATION, San Clemente, CA (US)

(72) Inventors: Timothy P. Murphy, San Clemente, CA (US); Harold Alexander Heitzmann, Irvine, CA (US)

(21) Appl. No.: 17/432,456

(22) PCT Filed: Feb. 21, 2020
(86) PCT No.: PCT/US2020/019345

§ 371 Date: Aug. 19, 2021

Related U.S. Application Data
(60) Provisional application No. 62/809,498, filed on Feb. 22, 2019.

(51) Int. Cl. C07D 217/02

(52) U.S. Cl. CPC C07D 217/02

(57) ABSTRACT

Disclosed herein are compounds for the treatment of ocular diseases and the preparation and use thereof. Some implementations relate to amine and sulfonamide derivatives and their use as therapeutic agents for the treatment of glaucoma.

### BACKGROUND

### Field

The present application relates to the fields of chemistry, biochemistry, and medicine. More particularly, this disclosure relates to compounds for the treatment of ocular diseases and disorders, and methods of synthesizing the same. In certain implementations, this disclosure relates to the treatment of glaucoma.

### Description of the Related Art

The mammalian eye is a specialized sensory organ capable of light reception and is able to receive visual images. The retina of the eye consists of photoreceptors that are sensitive to various levels of light, interneurons that relay signals from the photoreceptors to the retinal ganglion cells, which transmit the light-induced signals to the brain. The iris is an intraocular membrane that is involved in controlling the amount of light reaching the retina.

Numerous pathologies can compromise or entirely eliminate an individual's ability to perceive visual images, including trauma to the eye, infection, degeneration, vascular irregularities, and inflammatory problems. The central portion of the retina is known as the macula. The macula, which is responsible for central vision, fine visualization and color differentiation, may be affected by age related macular degeneration (wet or dry), diabetic macular edema, idiopathic choroidal neovascularization, or high myopia macular degeneration, among other pathologies.

About two percent of people in the United States have glaucoma, which is a group of eye diseases encompassing a broad spectrum of clinical presentations and etiologies but unified by increased intraocular pressure. Glaucoma causes pathological changes in the optic nerve, visible on the optic disk, and it causes corresponding visual field loss, which can result in blindness if untreated.

Glaucoma may be treated medically or surgically and may include the administration of one or more drugs, such as prostaglandins or prostaglandin analogs and Rho-kinase inhibitors.

### SUMMARY OF THE INVENTION

A compound of Formula (I), or a pharmaceutically acceptable salt, solvate, or prodrug thereof, having the structure:

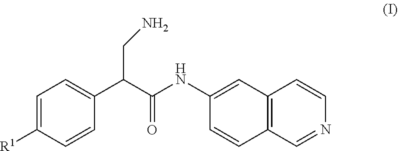

wherein R¹ is selected from the group consisting of hydrogen, an optionally substituted C₁-C₄ alkyl, and an optionally substituted naphthyl.

A compound of Formula (II), or a pharmaceutically acceptable salt, solvate, or prodrug thereof, having the structure:

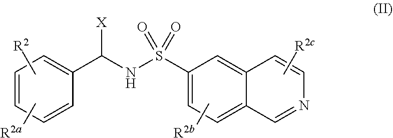

wherein:

X is selected from the group consisting of hydrogen, an optionally substituted C₁-C₄ alkyl, an optionally substituted C₂-C₄ alkenyl, an optionally substituted C₂-C₄ alkynyl, cyano, halogen, hydroxy, an optionally substituted aryl, and an optionally substituted aminoalkyl;

R² and R^{2a} are each independently selected from the group consisting of hydrogen, an optionally substituted C₁-C₄ alkyl, an optionally substituted C₂-C₄ alkenyl, an optionally substituted C₂-C₄ alkynyl, cyano, halogen, hydroxy, an optionally substituted aryl,

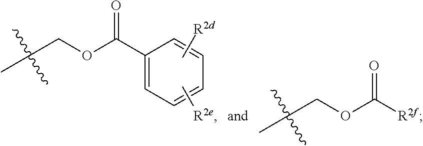

wherein R^{2d} and R^{2e} are each independently selected from the group consisting of hydrogen, an optionally substituted C₁-C₄ alkyl, an optionally substituted C₂-C₄ alkenyl, an optionally substituted C₂-C₄ alkynyl, cyano, halogen, hydroxy, and an optionally substituted aminoalkyl;

wherein R^{2f} is selected from the group consisting of an optionally substituted C₁-C₄ alkyl, an optionally substituted C₂-C₄ alkenyl, an optionally substituted C₂-C₄ alkynyl, an optionally substituted aryl, and an optionally substituted aralkyl; and

R^{2b} and R^{2c} are each independently selected from the group consisting of hydrogen, an optionally substituted C₁-C₄ alkyl, an optionally substituted C₂-C₄ alkenyl, an optionally substituted C₂-C₄ alkynyl, cyano, halogen, hydroxy, and an optionally substituted aminoalkyl.

A compound of Formula (III), or a pharmaceutically acceptable salt, solvate, or prodrug thereof, having the structure:

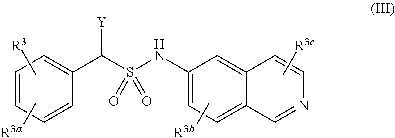

wherein:

Y is selected from the group consisting of hydrogen, an optionally substituted C₁-C₄ alkyl, an optionally substituted C₂-C₄ alkenyl, an optionally substituted C₂-C₄ alkynyl, cyano, halogen, hydroxy, an optionally substituted aryl, and an optionally substituted aminoalkyl;

R³ and R^{3a} are each independently selected from the group consisting of hydrogen, an optionally substituted C₁-C₄ alkyl, an optionally substituted C₂-C₄ alkenyl, an optionally substituted C₂-C₄ alkynyl, cyano, halogen, hydroxy, an optionally substituted aryl,

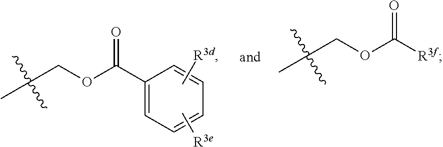

wherein R^{3d} and R^{3e} are each independently selected from the group consisting of hydrogen, an optionally substituted C₁-C₄ alkyl, an optionally substituted C₂-C₄ alkenyl, an optionally substituted C₂-C₄ alkynyl, cyano, halogen, hydroxy, and an optionally substituted aminoalkyl;

R^{3f} is selected from the group consisting of an optionally substituted C₁-C₄ alkyl, an optionally substituted C₂-C₄ alkenyl, an optionally substituted C₂-C₄ alkynyl, an optionally substituted aryl, and an optionally substituted aralkyl; and

R^{3b} and R^{3c} are each independently selected from the group consisting of hydrogen, an optionally substituted C₁-C₄ alkyl, an optionally substituted C₂-C₄ alkenyl, an optionally substituted C₂-C₄ alkynyl, cyano, halogen, hydroxy, and an optionally substituted aminoalkyl.

A compound of Formula (IV), or a pharmaceutically acceptable salt, solvate, or prodrug thereof, having the structure:

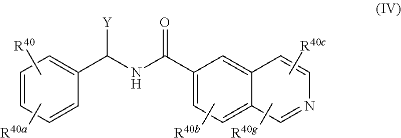

wherein:

Y is selected from the group consisting of hydrogen, an optionally substituted C₁-C₄ alkyl, an optionally substituted C₂-C₄ alkenyl, an optionally substituted C₂-C₄ alkynyl, cyano, halogen, hydroxy, an optionally substituted aryl, and an optionally substituted aminoalkyl;

R⁴⁰ and R^{40a} are each independently selected from the group consisting of hydrogen, an optionally substituted C₁-C₄ alkyl, an optionally substituted C₂-C₄ alkenyl, an optionally substituted C₂-C₄ alkynyl, cyano, halogen, hydroxy, an optionally substituted aryl,

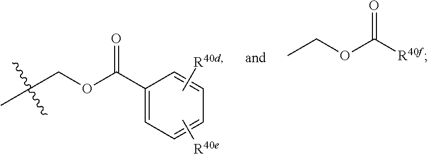

wherein R^{40d} and R^{40e} are each independently selected from the group consisting of hydrogen, an optionally substituted C₁-C₄ alkyl, an optionally substituted C₂-C₄ alkenyl, an optionally substituted C₂-C₄ alkynyl, cyano, halogen, hydroxy, and an optionally substituted aminoalkyl;

R^{40f} is selected from the group consisting of an optionally substituted C₁-C₄ alkyl, an optionally substituted C₂-C₄ alkenyl, an optionally substituted C₂-C₄ alkynyl, an optionally substituted aryl, and an optionally substituted aralkyl; and

R^{40b}, R^{40c}, and R^{40g} are each independently selected from the group consisting of hydrogen, an optionally substituted C₁-C₄ alkyl, an optionally substituted C₂-C₄ alkenyl, an optionally substituted C₂-C₄ alkynyl, cyano, halogen, hydroxy, an optionally substituted aminoalkyl, and a keto-enol tautomer.

Other implementations disclosed herein include a pharmaceutical composition comprising a therapeutically effective amount of a compound disclosed herein and a pharmaceutically acceptable excipient.

### DETAILED DESCRIPTION

In some implementations, compounds that contain an amide or sulfonamide moiety are provided that act as agents used in the treatment of ocular diseases. Various implementations of these compounds include compounds of Formula (I), Formula (II), Formula (III), and Formula (IV), or pharmaceutically acceptable salts, solvates, or prodrugs thereof.

It is believed that the compounds disclosed herein are active against glaucoma as Rho-kinase inhibitors, although these compounds may be effective via other routes of action.

In some implementations, compounds of Formula (I) include compounds in which R¹ is selected from the group consisting of hydrogen, an optionally substituted C₁-C₄ alkyl, and an optionally substituted naphthyl. In some implementations, R¹ is —CH₂OH.

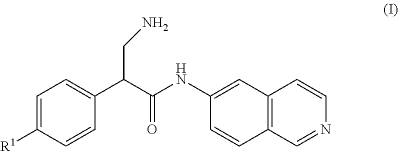

In some implementations, compounds of Formula (II) include compounds in which R^{2b} and R^{2c} are each selected from the group consisting of hydrogen, an optionally substituted C₁-C₄ alkyl, an optionally substituted C₂-C₄ alkenyl, an optionally substituted C₂-C₄ alkynyl, cyano, halogen, hydroxy, and an optionally substituted aminoalkyl.

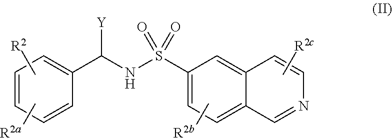

In some implementations, R^{2b} and R^{2c} are each independently selected from the group consisting of hydrogen, hydroxy, and —CH₂CH₂NH₂.

In some implementations of Formula (II), R^{2b} and R^{2C} are both hydrogen.

In some implementations, compounds of Formula (II) include compounds in which X is selected from the group consisting of hydrogen, an optionally substituted C₁-C₄ alkyl, an optionally substituted C₂-C₄ alkenyl, an optionally substituted C₂-C₄ alkynyl, cyano, halogen, hydroxy, an optionally substituted aryl, and an optionally substituted aminoalkyl.

In some implementations of Formula (II), X is an optionally substituted aminoalkyl.

In some implementations of Formula (II), X is —CH₂NH₂.

In some implementations, compounds of Formula (II) include compounds in which R² and R^{2a} are each independently selected from the group consisting of hydrogen, an optionally substituted C₁-C₄ alkyl, an optionally substituted C₂-C₄ alkenyl, an optionally substituted C₂-C₄ alkynyl, cyano, halogen, hydroxy, an optionally substituted aryl,

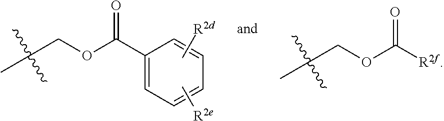

In some implementations of Formula (II), R² and R^{2a} are not both hydrogen.

In some implementations of Formula (II), R² is hydrogen and R^{2a} is selected from the group consisting of methyl, —CH₂OH, —Cl, naphthyl, and

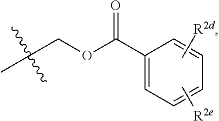

wherein R^{2d} and R^{2e} are each independently selected from the group consisting of hydrogen, an optionally substituted C₁-C₄ alkyl, an optionally substituted C₂-C₄ alkenyl, an optionally substituted C₂-C₄ alkynyl, cyano, halogen, hydroxy, and an optionally substituted aminoalkyl. In some implementations, R^{2d} and R^{2e} are each independently selected from the group consisting of hydrogen, methyl, and —CH₂CH₂NH₂.

In some implementations, R^{2f} is selected from the group consisting of an optionally substituted C₁-C₄ alkyl, an optionally substituted C₂-C₄ alkenyl, an optionally substituted C₂-C₄ alkynyl, an optionally substituted aryl, and an optionally substituted aralkyl.

Some implementations of Formula (II) include a compound selected from the group consisting of:

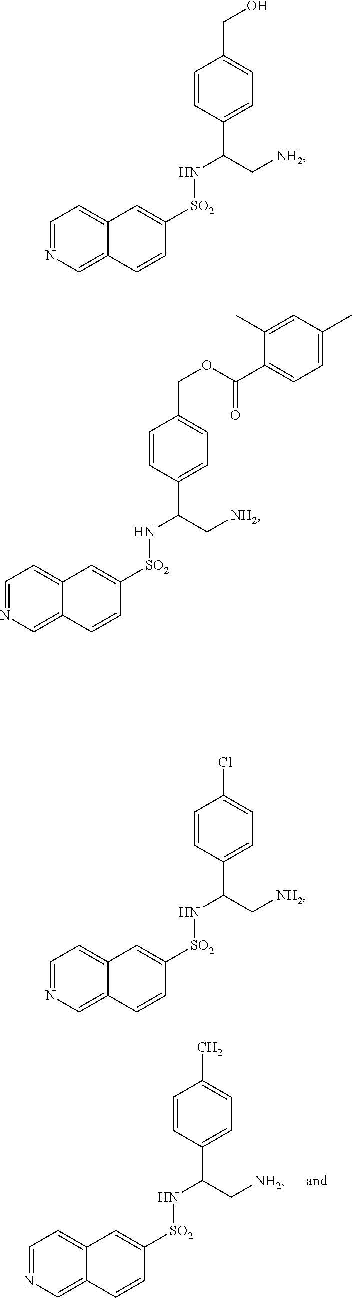 [SMILES: NCC(NS(=O)(=O)c1ccc2cnccc2c1)c1ccc(CO)cc1 ; Cc1ccc(C(=O)OCc2ccc(C(CN)NS(=O)(=O)c3ccc4cnccc4c3)cc2)c(C)c1 ; NCC(NS(=O)(=O)c1ccc2cnccc2c1)c1ccc(Cl)cc1 ; Cc1ccc(C(CN)NS(=O)(=O)c2ccc3cnccc3c2)cc1]

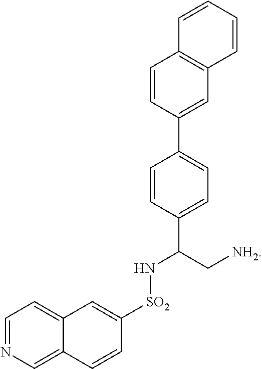 [SMILES: NCC(NS(=O)(=O)c1ccc2cnccc2c1)c1ccc(-c2ccc3ccccc3c2)cc1]

In some implementations, compounds of Formula (III) include compounds in which R^{3b} and R^{3c} are each selected from the group consisting of hydrogen, an optionally substituted C₁-C₄ alkyl, an optionally substituted C₂-C₄ alkenyl, an optionally substituted C₂-C₄ alkynyl, cyano, halogen, hydroxy, and an optionally substituted aminoalkyl.

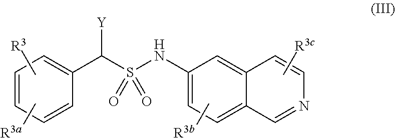

In some implementations of Formula (III), R^{3b} and R^{3c} are each independently selected from the group consisting of hydrogen, hydroxy, and —CH₂CH₂NH₂.

In some implementations of Formula (III), R^{3b} and R^{3c} are both hydrogen.

In some implementations, compounds of Formula (III) include compounds in which Y is selected from the group consisting of hydrogen, an optionally substituted C₁-C₄ alkyl, an optionally substituted C₂-C₄ alkenyl, an optionally substituted C₂-C₄ alkynyl, cyano, halogen, hydroxy, an optionally substituted aryl, and an optionally substituted aminoalkyl.

In some implementations of Formula (III), Y is an optionally substituted aminoalkyl.

In some implementations of Formula (III), Y is —CH₂NH₂.

In some implementations of Formula (III), R³ and R^{3a} are each independently selected from the group consisting of hydrogen, an optionally substituted C₁-C₄ alkyl, an optionally substituted C₂-C₄ alkenyl, an optionally substituted C₂-C₄ alkynyl, cyano, halogen, hydroxy, an optionally substituted aryl

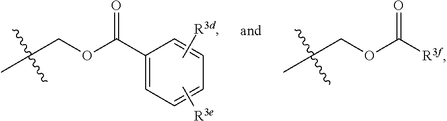

wherein R³ and R^{3a} are not both hydrogen, and R^{3d} and R^{3c} are each independently selected from the group consisting of hydrogen, an optionally substituted C₁-C₄ alkyl, an optionally substituted C₂-C₄ alkenyl, an optionally substituted C₂-C₄ alkynyl, cyano, halogen, hydroxy, and an optionally substituted aminoalkyl.

In some implementations, R^{3f} is selected from the group consisting of an optionally substituted C₁-C₄ alkyl, an optionally substituted C₂-C₄ alkenyl, an optionally substituted C₂-C₄ alkynyl, an optionally substituted aryl, and an optionally substituted aralkyl.

In some implementations of Formula (III), R³ and R^{3a} are each independently selected from the group consisting of hydrogen, an optionally substituted C₁-C₄ alkyl, halogen, an optionally substituted aryl, and

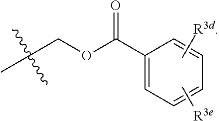

In some implementations of Formula (III), R³ is hydrogen and R^{3a} is selected from the group consisting of methyl, —CH₂OH, —Cl, naphthyl, and

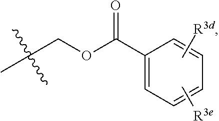

wherein R^{3d} and R^{3c} are each independently selected from the group consisting of hydrogen, an optionally substituted C₁-C₄ alkyl, an optionally substituted C₂-C₄ alkenyl, an optionally substituted C₂-C₄ alkynyl, cyano, halogen, hydroxy, and an optionally substituted aminoalkyl. In some implementations, R^{3d} and R^{3c} are each independently selected from the group consisting of hydrogen, methyl, and —CH₂CH₂NH₂.

Some implementations of Formula (III) include a compound selected from the group consisting of:

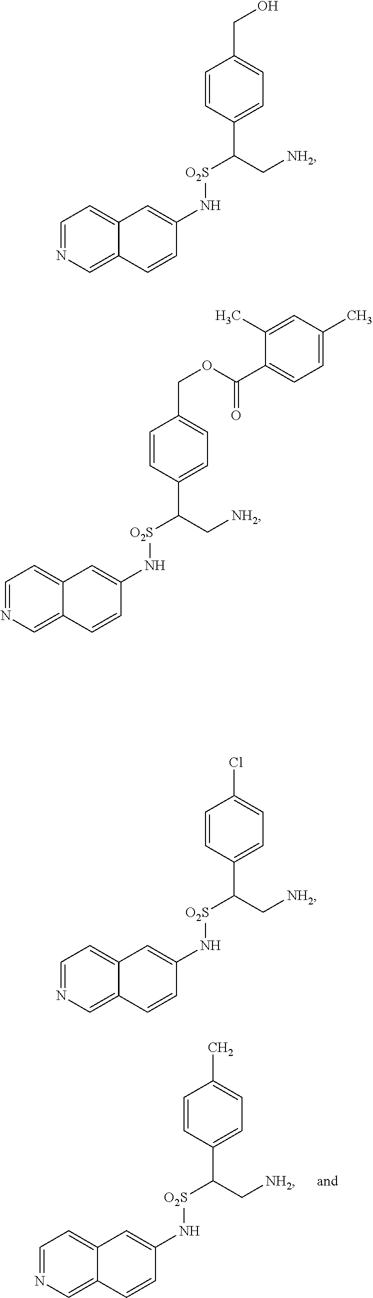 [SMILES: NCC(c1ccc(CO)cc1)S(=O)(=O)Nc1ccc2cnccc2c1 ; Cc1ccc(C(=O)OCc2ccc(C(CN)S(=O)(=O)Nc3ccc4cnccc4c3)cc2)c(C)c1 ; NCC(c1ccc(Cl)cc1)S(=O)(=O)Nc1ccc2cnccc2c1 ; Cc1ccc(C(CN)S(=O)(=O)Nc2ccc3cnccc3c2)cc1]

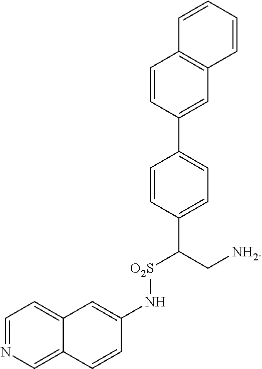 [SMILES: NCC(c1ccc(-c2ccc3ccccc3c2)cc1)S(=O)(=O)Nc1ccc2cnccc2c1]

In some implementations, compounds of Formula (IV) include compounds in which R^{40b}, R^{40c} and R^{40g} are each selected from the group consisting of hydrogen, an , optionally substituted C₁-C₄ alkyl, an optionally substituted C₂-C₄ alkenyl, an optionally substituted C₂-C₄ alkynyl, cyano, halogen, hydroxy, and an optionally substituted aminoalkyl.

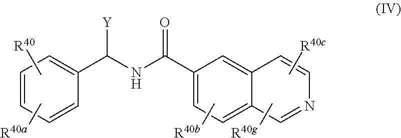

In some implementations of Formula (IV), R^{40b}, R^{40c}, and R^{40g} are each independently selected from the group consisting of hydrogen, hydroxy, and —CH₂CH₂NH₂.

In some implementations of Formula (IV), R^{40b}, R^{40c}, and R^{40g} are all hydrogen.

In some implementations, compounds of Formula (IV) include compounds in which Y is selected from the group consisting of hydrogen, an optionally substituted C₁-C₄ alkyl, an optionally substituted C₂-C₄ alkenyl, an optionally substituted C₂-C₄ alkynyl, cyano, halogen, hydroxy, an optionally substituted aryl, and an optionally substituted aminoalkyl.

In some implementations of Formula (IV), Y is an optionally substituted aminoalkyl.

In some implementations of Formula (IV), Y is —CH₂NH₂.

In some implementations of Formula (IV), R⁴⁰ and R^{40a} are each independently selected from the group consisting of hydrogen, an optionally substituted C₁-C₄ alkyl, an optionally substituted C₂-C₄ alkenyl, an optionally substituted C₂-C₄ alkynyl, cyano, halogen, hydroxy, an optionally substituted aryl,

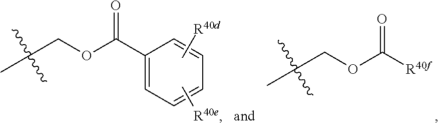

wherein R⁴⁰ and R^{40a} are not both hydrogen, and R^{40a} and R^{40e} are each independently selected from the group consisting of hydrogen, an optionally substituted C₁-C₄ alkyl, an optionally substituted C₂-C₄ alkenyl, an optionally substituted C₂-C₄ alkynyl, cyano, halogen, hydroxy, and an optionally substituted aminoalkyl. In some implementations, R^{40f} is selected from the group consisting of an optionally substituted C₁-C₄ alkyl, an optionally substituted C₂-C₄ alkenyl, an optionally substituted C₂-C₄ alkynyl, an optionally substituted aryl, and an optionally substituted aralkyl.

In some implementations of Formula (IV), R⁴⁰ and R^{40a} are each independently selected from the group consisting of hydrogen, an optionally substituted C₁-C₄ alkyl, halogen, an optionally substituted aryl, and

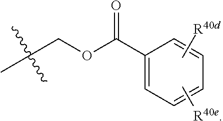

In some implementations of Formula (IV), R⁴⁰ is hydrogen and R^{40a} is selected from the group consisting of methyl, —CH₂OH, —Cl, naphthyl, and

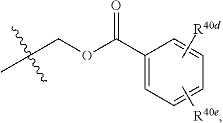

wherein R^{40d} and R^{40e} are each independently selected from the group consisting of hydrogen, an optionally substituted C₁-C₄ alkyl, an optionally substituted C₂-C₄ alkenyl, an optionally substituted C₂-C₄ alkynyl, cyano, halogen, hydroxy, and an optionally substituted aminoalkyl. In some implementations, R^{40d} and R^{40e} are each independently selected from the group consisting of hydrogen, methyl, and —CH₂CH₂NH₂.

Some implementations of Formula (IV) include a compound selected from the group consisting of:

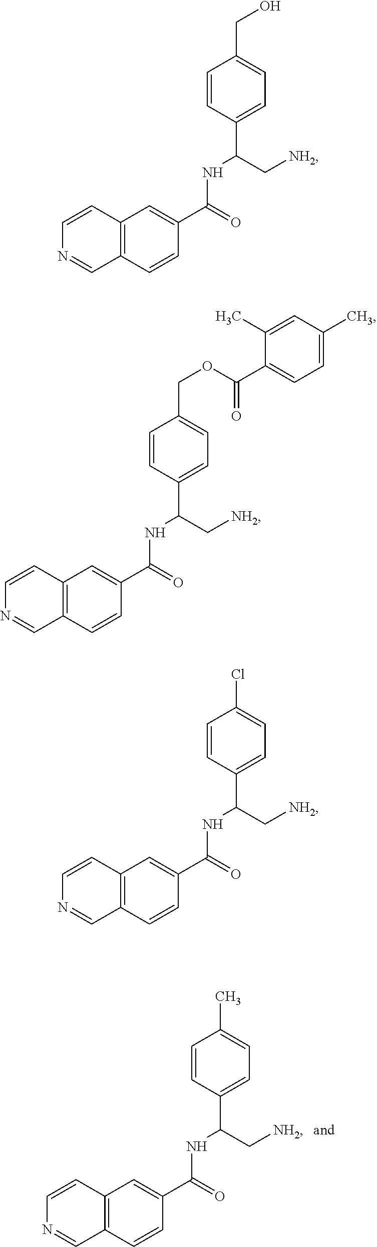 [SMILES: NCC(NC(=O)c1ccc2cnccc2c1)c1ccc(CO)cc1 ; Cc1ccc(C(=O)OCc2ccc(C(CN)NC(=O)c3ccc4cnccc4c3)cc2)c(C)c1 ; NCC(NC(=O)c1ccc2cnccc2c1)c1ccc(Cl)cc1 ; Cc1ccc(C(CN)NC(=O)c2ccc3cnccc3c2)cc1]

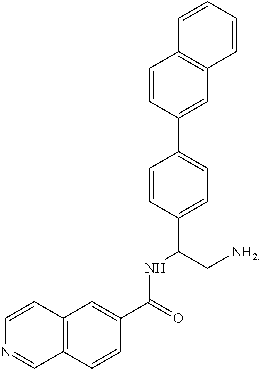 [SMILES: NCC(NC(=O)c1ccc2cnccc2c1)c1ccc(-c2ccc3ccccc3c2)cc1]

In some implementations, the compounds of Formula (I), (II), (III), or (IV) exist as a prodrug.

### Definitions

Unless defined otherwise, all technical and scientific terms used herein have the same meaning as is commonly understood by one of ordinary skill in the art. All patents, applications, published applications, and other publications referenced herein are incorporated by reference in their entirety unless stated otherwise. In the event that there are a plurality of definitions for a term herein, those in this section prevail unless stated otherwise.

A “prodrug” refers to an agent that is converted into the parent drug in vivo. Prodrugs are often useful because, in some situations, they may be easier to administer than the parent drug. They may, for instance, be bioavailable by oral administration whereas the parent is not. The prodrug may also have improved solubility in pharmaceutical compositions over the parent drug. An example, without limitation, of a prodrug would be a compound which is administered as an ester (the “prodrug”) to facilitate transmittal across a cell membrane where water solubility is detrimental to mobility but which then is metabolically hydrolyzed to the carboxylic acid, the active entity, once inside the cell where water-solubility is beneficial. A further example of a prodrug might be a short peptide (polyaminoacid) bonded to an acid group where the peptide is metabolized to reveal the active moiety. Conventional procedures for the selection and preparation of suitable prodrug derivatives are described, for example, in Design of Prodrugs, (ed. H. Bundgaard, Elsevier, 1985), which is hereby incorporated herein by reference in its entirety.

The term “pharmaceutically acceptable carrier” or “pharmaceutically acceptable excipient” includes any and all solvents, dispersion media, coatings, antibacterial and antifungal agents, antioxidants, isotonic and absorption delaying agents and the like. The use of such media and agents for pharmaceutically active substances is well known in the art. Except insofar as any conventional media or agent is incompatible with the active ingredient, its use in the therapeutic compositions is contemplated. In addition, various adjuvants such as are commonly used in the art may be included. Considerations for the inclusion of various components in pharmaceutical compositions are described, e.g., in Gilman et al. (Eds.) (1990); Goodman and Gilman's: The Pharmacological Basis of Therapeutics, 8th Ed., Pergamon Press, which is incorporated herein by reference in its entirety.

“Treat,” “treatment,” or “treating,” as used herein refers to administering a pharmaceutical composition for prophylactic and/or therapeutic purposes. The term “prophylactic treatment” refers to treating a subject who does not yet exhibit symptoms of a disease or condition, but who is susceptible to, or otherwise at risk of, a particular disease or condition, whereby the treatment reduces the likelihood that the patient will develop the disease or condition.

Whenever a group is described as being “substituted” or “optionally substituted” that group may be substituted with one or more of the indicated substituents. If no substituents are indicated, it is meant that the indicated “optionally substituted” or “substituted” group may be substituted with one or more group(s) individually and independently selected from alkyl, alkenyl, alkynyl, cycloalkyl, cycloalkenyl, acylalkyl, hydroxy, alkoxy, alkoxyalkyl, aminoalkyl, amino acid, aryl, heteroaryl, heterocyclyl, aryl(alkyl), heteroaryl(alkyl), heterocyclyl(alkyl), hydroxyalkyl, acyl, cyano, halogen, thiocarbonyl, O-carbamyl, N-carbamyl, O-thiocarbamyl, N-thiocarbamyl, C-amido, N-amido, S-sulfonamido, N-sulfonamido, C-carboxy, O-carboxy, isocyanato, thiocyanato, isothiocyanato, azido, nitro, silyl, sulfenyl, sulfinyl, sulfonyl, naphthyl, haloalkyl, haloalkoxy, trihalomethanesulfonyl, trihalomethanesulfonamido, an amino, a mono-substituted amino, and a di-substituted amino.

As used herein, “alkyl” refers to a straight or branched hydrocarbon chain that comprises a fully saturated (no double or triple bonds) hydrocarbon group. The alkyl group may have 1 to 20 carbon atoms (whenever it appears herein, a numerical range such as “1 to 20” refers to each integer in the given range; e.g., “1 to 20 carbon atoms” means that the alkyl group may consist of 1 carbon atom, 2 carbon atoms, 3 carbon atoms, etc., up to and including 20 carbon atoms, although the present definition also covers the occurrence of the term “alkyl” where no numerical range is designated). The alkyl group may also be a medium size alkyl having 1 to 10 carbon atoms. The alkyl group could also be a lower alkyl having 1 to 6 carbon atoms. The alkyl group of the compounds may be designated as “C1-C4 alkyl” or similar designations. By way of example only, “C1-C4 alkyl” indicates that there are one to four carbon atoms in the alkyl chain, i.e., the alkyl chain is selected from methyl, ethyl, propyl, iso-propyl, n-butyl, iso-butyl, sec-butyl, and t-butyl. Typical alkyl groups include, but are in no way limited to, methyl, ethyl, propyl, isopropyl, butyl, isobutyl, tertiary butyl, pentyl and hexyl. The alkyl group may be substituted or unsubstituted.

As used herein, “alkenyl” refers to an alkyl group that contains in the straight or branched hydrocarbon chain one or more double bonds. Examples of alkenyl groups include allenyl, vinylmethyl and ethenyl. An alkenyl group may be unsubstituted or substituted.

As used herein, “alkynyl” refers to an alkyl group that contains in the straight or branched hydrocarbon chain one or more triple bonds. Examples of alkynyls include ethynyl and propynyl. An alkynyl group may be unsubstituted or substituted.

As used herein, “cycloalkyl” refers to a completely saturated (no double or triple bonds) mono- or multi-cyclic hydrocarbon ring system. When composed of two or more rings, the rings may be joined together in a fused fashion. Cycloalkyl groups can contain 3 to 10 atoms in the ring(s) or 3 to 8 atoms in the ring(s). A cycloalkyl group may be unsubstituted or substituted. Typical cycloalkyl groups include, but are in no way limited to, cyclopropyl, cyclobutyl, cyclopentyl, cyclohexyl, cycloheptyl and cyclooctyl.

As used herein, “cycloalkenyl” refers to a mono- or multi-cyclic hydrocarbon ring system that contains one or more double bonds in at least one ring; although, if there is more than one, the double bonds cannot form a fully delocalized pi-electron system throughout all the rings (otherwise the group would be “aryl,” as defined herein). Cycloalkenyl groups can contain 3 to 10 atoms in the ring(s) or 3 to 8 atoms in the ring(s). When composed of two or more rings, the rings may be connected together in a fused fashion. A cycloalkenyl group may be unsubstituted or substituted.

As used herein, “aryl” refers to a carbocyclic (all carbon) monocyclic or multicyclic aromatic ring system (including fused ring systems where two carbocyclic rings share a chemical bond) that has a fully delocalized pi-electron system throughout all the rings. The number of carbon atoms in an aryl group can vary. For example, the aryl group can be a C₆-C₁₄ aryl group, a C₆-C₁₀ aryl group, or a C₆ aryl group. Examples of aryl groups include, but are not limited to, phenyl and naphthyl. An aryl group may be substituted or unsubstituted.

As used herein, “heteroaryl” refers to a monocyclic or multicyclic aromatic ring system (a ring system with fully delocalized pi-electron system) that contain(s) one, two, three or more heteroatoms, that is, an element other than carbon, including but not limited to, nitrogen, oxygen and sulfur. The number of atoms in the ring(s) of a heteroaryl group can vary. For example, the heteroaryl group can contain 4 to 14 atoms in the ring(s), 5 to 10 atoms in the ring(s) or 5 to 6 atoms in the ring(s). Furthermore, the term “heteroaryl” includes fused ring systems where two rings, such as at least one aryl ring and at least one heteroaryl ring, or at least two heteroaryl rings, share at least one chemical bond. Examples of heteroaryl rings include, but are not limited to, those described herein and the following: furan, furazan, thiophene, benzothiophene, phthalazine, pyrrole, oxazole, benzoxazole, 1,2,3-oxadiazole, 1,2,4-oxadiazole, thiazole, 1,2,3-thiadiazole, 1,2,4-thiadiazole, benzothiazole, imidazole, benzimidazole, indole, indazole, pyrazole, benzopyrazole, isoxazole, benzoisoxazole, isothiazole, triazole, benzotriazole, thiadiazole, tetrazole, pyridine, pyridazine, pyrimidine, pyrazine, purine, pteridine, quinoline, isoquinoline, quinazoline, quinoxaline, cinnoline and triazine. A heteroaryl group may be substituted or unsubstituted.

As used herein, “heterocyclyl” or “heteroalicyclyl” refers to three-, four-, five-, six-, seven-, eight-, nine-, ten-, up to 18-membered monocyclic, bicyclic, and tricyclic ring system wherein carbon atoms together with from 1 to 5 heteroatoms constitute said ring system. A heterocycle may optionally contain one or more unsaturated bonds situated in such a way, however, that a fully delocalized pi-electron system does not occur throughout all the rings. The heteroatom(s) is an element other than carbon including, but not limited to, oxygen, sulfur, and nitrogen. A heterocycle may further contain one or more carbonyl or thiocarbonyl functionalities, so as to make the definition include oxo-systems and thio-systems such as lactams, lactones, cyclic imides, cyclic thioimides and cyclic carbamates. When composed of two or more rings, the rings may be joined together in a fused fashion. Additionally, any nitrogens in a heterocyclyl may be quaternized. Heterocyclyl or heteroalicyclic groups may be unsubstituted or substituted. Examples of such “heterocyclyl” or “heteroalicyclyl” groups include, but are not limited to, those described herein and the following: 1,3-dioxin, 1,3-dioxane, 1,4-dioxane, 1,2-dioxolane, 1,3-dioxolane, 1,4-dioxolane, 1,3-oxathiane, 1,4-oxathiin, 1,3-oxathiolane, 1,3-dithiole, 1,3-dithiolane, 1,4-oxathiane, tetrahydro-1,4-thiazine, 1,3-thiazinane, 2H-1,2-oxazine, maleimide, succinimide, barbituric acid, thiobarbituric acid, dioxopiperazine, hydantoin, dihydrouracil, trioxane, hexahydro-1,3,5-triazine, imidazoline, imidazolidine, isoxazoline, isoxazolidine, oxazoline, oxazolidine, oxazolidinone, thiazoline, thiazolidine, morpholine, oxirane, piperidine N-Oxide, piperidine, piperazine, pyrrolidine, pyrrolidone, pyrrolidione, 4-piperidone, pyrazoline, pyrazolidine, 2-oxopyrrolidine, tetrahydropyran, 4H-pyran, tetrahydrothiopyran, thiamorpholine, thiamorpholine sulfoxide, thiamorpholine sulfone, and their benzo-fused analogs (e.g., benzimidazolidinone, tetrahydroquinoline, and 3,4-methylenedioxyphenyl).

As used herein, “aralkyl” and “aryl(alkyl)” refer to an aryl group connected, as a substituent, via a lower alkylene group. The lower alkylene and aryl group of an aralkyl may be substituted or unsubstituted. Examples include but are not limited to benzyl, xylyl, tolyl, 2-phenylalkyl, 3-phenylalkyl and naphthylalkyl.

As used herein, “heteroaralkyl” and “heteroaryl(alkyl)” refer to a heteroaryl group connected, as a substituent, via a lower alkylene group. The lower alkylene and heteroaryl group of heteroaralkyl may be substituted or unsubstituted. Examples include but are not limited to 2-thienylalkyl, 3-thienylalkyl, furylalkyl, thienylalkyl, pyrrolylalkyl, pyridylalkyl, isoxazolylalkyl, imidazolylalkyl and their benzo-fused analogs.

A “heteroalicyclyl(alkyl)” and “heterocyclyl(alkyl)” refer to a heterocyclic or a heteroalicyclylic group connected, as a substituent, via a lower alkylene group. The lower alkylene and heterocyclyl of a heteroalicyclyl(alkyl) may be substituted or unsubstituted. Examples include but are not limited tetrahydro-2H-pyran-4-yl(methyl), piperidin-4-yl(ethyl), piperidin-4-yl(propyl), tetrahydro-2H-thiopyran-4-yl(methyl), and 1,3-thiazinan-4-yl(methyl).

“Lower alkylene groups” are straight-chained —CH₂— tethering groups, forming bonds to connect molecular fragments via their terminal carbon atoms. Examples include but are not limited to methylene (—CH₂—), ethylene (—CH₂CH₂—), propylene (—CH₂CH₂CH₂—), and butylene (—CH₂CH₂CH₂CH₂—). A lower alkylene group can be substituted by replacing one or more hydrogen of the lower alkylene group with a substituent(s) listed under the definition of “substituted.”

As used herein, “alkoxy” refers to the formula —OR wherein R is an alkyl, an alkenyl, an alkynyl, a cycloalkyl, a cycloalkenyl, aryl, heteroaryl, heterocyclyl, cycloalkyl(alkyl), aryl(alkyl), heteroaryl(alkyl) or heterocyclyl(alkyl) is defined herein. A non-limiting list of alkoxys are methoxy, ethoxy, n-propoxy, 1-methylethoxy (isopropoxy), n-butoxy, iso-butoxy, sec-butoxy, tert-butoxy, phenoxy and benzoxy. An alkoxy may be substituted or unsubstituted.

As used herein, “acyl” refers to a hydrogen, an alkyl, an alkenyl, an alkynyl, a cycloalkyl, a cycloalkenyl, aryl, heteroaryl, heterocyclyl, cycloalkyl(alkyl), aryl(alkyl), heteroaryl(alkyl) or heterocyclyl(alkyl) connected, as substituents, via a carbonyl group. Examples include formyl, acetyl, propanoyl, benzoyl and acryl. An acyl may be substituted or unsubstituted.

As used herein, “acylalkyl” refers to an acyl connected, as a substituent, via a lower alkylene group. Examples include aryl-C(═O)—(CH₂)ₙ— and heteroaryl-C(═O)—(CH₂)ₙ—, where n is an integer in the range of 1 to 6.

As used herein, “alkoxyalkyl” refers to an alkoxy group connected, as a substituent, via a lower alkylene group. Examples include C₁₋₄ alkyl-O—(CH₂)ₙ—, wherein n is an integer in the range of 1 to 6.

As used herein, “aminoalkyl” refers to an optionally substituted amino group connected, as a substituent, via a lower alkylene group. Examples include —CH₂NH₂ and —CH₂CH₂NH₂. The nitrogen in the aminoalkyl group may itself be substituted by one or two additional lower alkylene groups.

As used herein, “hydroxyalkyl” refers to an alkyl group in which one or more of the hydrogen atoms are replaced by a hydroxy group. Exemplary hydroxyalkyl groups include but are not limited to, 2-hydroxyethyl, 3-hydroxypropyl, 2-hydroxypropyl, and 2,2-dihydroxyethyl. A hydroxyalkyl may be substituted or unsubstituted.

As used herein, “haloalkyl” refers to an alkyl group in which one or more of the hydrogen atoms are replaced by a halogen (e.g., mono-haloalkyl, di-haloalkyl and tri-haloalkyl). Such groups include but are not limited to, chloromethyl, fluoromethyl, difluoromethyl, trifluoromethyl, chloro-fluoroalkyl, chloro-difluoroalkyl and 2-fluoroisobutyl. A haloalkyl may be substituted or unsubstituted.

As used herein, “haloalkoxy” refers to an alkoxy group in which one or more of the hydrogen atoms are replaced by a halogen (e.g., mono-haloalkoxy, di-haloalkoxy and tri-haloalkoxy). Such groups include but are not limited to, chloromethoxy, fluoromethoxy, difluoromethoxy, trifluoromethoxy, chloro-fluoroalkyl, chloro-difluoroalkoxy and 2-fluoroisobutoxy. A haloalkoxy may be substituted or unsubstituted.

A “sulfenyl” group refers to an “—SR” group in which R can be hydrogen, an alkyl, an alkenyl, an alkynyl, a cycloalkyl, a cycloalkenyl, aryl, heteroaryl, heterocyclyl, cycloalkyl(alkyl), aryl(alkyl), heteroaryl(alkyl) or heterocyclyl(alkyl). A sulfenyl may be substituted or unsubstituted.

A “sulfinyl” group refers to an “—S(═O)—R” group in which R can be the same as defined with respect to sulfenyl. A sulfinyl may be substituted or unsubstituted.

A “sulfonyl” group refers to an “—SO₂R” group in which R can be the same as defined with respect to sulfenyl. A sulfonyl may be substituted or unsubstituted.

An “O-carboxy” group refers to a “RC(═O)O—” group in which R can be hydrogen, an alkyl, an alkenyl, an alkynyl, a cycloalkyl, a cycloalkenyl, aryl, heteroaryl, heterocyclyl, cycloalkyl(alkyl), aryl(alkyl), heteroaryl(alkyl) or heterocyclyl(alkyl), as defined herein. An O-carboxy may be substituted or unsubstituted.

The terms “ester” and “C-carboxy” refer to a “—C(═O)OR” group in which R can be the same as defined with respect to O-carboxy. An ester and C-carboxy may be substituted or unsubstituted.

A “thiocarbonyl” group refers to a “—C(═S)R” group in which R can be the same as defined with respect to O-carboxy. A thiocarbonyl may be substituted or unsubstituted.

A “trihalomethanesulfonyl” group refers to an “X₃CSO₂—” group wherein each X is a halogen.

A “trihalomethanesulfonamido” group refers to an “X₃CS(O)₂N(R_{A})—” group wherein each X is a halogen, and R_{A} hydrogen, an alkyl, an alkenyl, an alkynyl, a cycloalkyl, a cycloalkenyl, aryl, heteroaryl, heterocyclyl, cycloalkyl(alkyl), aryl(alkyl), heteroaryl(alkyl) or heterocyclyl(alkyl).

The term “amino” as used herein refers to a —NH₂ group.

As used herein, the term “hydroxy” refers to a —OH group.

A “cyano” group refers to a “—CN” group.

The term “azido” as used herein refers to a —N₃ group.

An “isocyanato” group refers to a “—NCO” group.

A “thiocyanato” group refers to a “—CNS” group.

An “isothiocyanato” group refers to an “—NCS” group.

A “carbonyl” group refers to a C═O group.

An “S-sulfonamido” group refers to a “—SO₂N(R_{A}R_{B})” group in which R_{A} and R_{B} can be independently hydrogen, an alkyl, an alkenyl, an alkynyl, a cycloalkyl, a cycloalkenyl, aryl, heteroaryl, heterocyclyl, cycloalkyl(alkyl), aryl(alkyl), heteroaryl(alkyl) or heterocyclyl(alkyl). An S-sulfonamido may be substituted or unsubstituted.

An “N-sulfonamido” group refers to a “RSO₂N(R_{A})—” group in which R and R_{A} can be independently hydrogen, an alkyl, an alkenyl, an alkynyl, a cycloalkyl, a cycloalkenyl, aryl, heteroaryl, heterocyclyl, cycloalkyl(alkyl), aryl(alkyl), heteroaryl(alkyl) or heterocyclyl(alkyl). An N-sulfonamido may be substituted or unsubstituted.

An “O-carbamyl” group refers to a “—OC(═O)N(R_{A}R_{B})” group in which R_{A} and R_{B} can be independently hydrogen, an alkyl, an alkenyl, an alkynyl, a cycloalkyl, a cycloalkenyl, aryl, heteroaryl, heterocyclyl, cycloalkyl(alkyl), aryl(alkyl), heteroaryl(alkyl) or heterocyclyl(alkyl). An O-carbamyl may be substituted or unsubstituted.

An “N-carbamyl” group refers to an “ROC(═O)N(R_{A})—” group in which R and R_{A} can be independently hydrogen, an alkyl, an alkenyl, an alkynyl, a cycloalkyl, a cycloalkenyl, aryl, heteroaryl, heterocyclyl, cycloalkyl(alkyl), aryl(alkyl), heteroaryl(alkyl) or heterocyclyl(alkyl). An N-carbamyl may be substituted or unsubstituted.

An “O-thiocarbamyl” group refers to a “—OC(═S)—N(R_{A}R_{B})” group in which R_{A} and R_{B} can be independently hydrogen, an alkyl, an alkenyl, an alkynyl, a cycloalkyl, a cycloalkenyl, aryl, heteroaryl, heterocyclyl, cycloalkyl(alkyl), aryl(alkyl), heteroaryl(alkyl) or heterocyclyl(alkyl). An O-thiocarbamyl may be substituted or unsubstituted.

An “N-thiocarbamyl” group refers to an “ROC(═S)N(R_{A})—” group in which R and R_{A} can be independently hydrogen, an alkyl, an alkenyl, an alkynyl, a cycloalkyl, a cycloalkenyl, aryl, heteroaryl, heterocyclyl, cycloalkyl(alkyl), aryl(alkyl), heteroaryl(alkyl) or heterocyclyl(alkyl). An N-thiocarbamyl may be substituted or unsubstituted.

A “C-amido” group refers to a “—C(═O)N(R_{A}R_{B})” group in which R_{A} and R_{B} can be independently hydrogen, an alkyl, an alkenyl, an alkynyl, a cycloalkyl, a cycloalkenyl, aryl, heteroaryl, heterocyclyl, cycloalkyl(alkyl), aryl(alkyl), heteroaryl(alkyl) or heterocyclyl(alkyl). A C-amido may be substituted or unsubstituted.

An “N-amido” group refers to a “RC(═O)N(R_{A})—” group in which R and R_{A} can be independently hydrogen, an alkyl, an alkenyl, an alkynyl, a cycloalkyl, a cycloalkenyl, aryl, heteroaryl, heterocyclyl, cycloalkyl(alkyl), aryl(alkyl), heteroaryl(alkyl) or heterocyclyl(alkyl). An N-amido may be substituted or unsubstituted.

The term “halogen atom” or “halogen” as used herein, means any one of the radio-stable atoms of column 7 of the Periodic Table of the Elements, such as, fluorine, chlorine, bromine and iodine.

The terms “protecting group” and “protecting groups” as used herein refer to any atom or group of atoms that is added to a molecule in order to prevent existing groups in the molecule from undergoing unwanted chemical reactions. Examples of protecting group moieties are described in T. W. Greene and P. G. M. Wuts, Protective Groups in Organic Synthesis, 3. Ed. John Wiley & Sons, 1999, and in J. F. W. McOmie, Protective Groups in Organic Chemistry Plenum Press, 1973, both of which are hereby incorporated by reference for the limited purpose of disclosing suitable protecting groups. The protecting group moiety may be chosen in such a way, that they are stable to certain reaction conditions and readily removed at a convenient stage using methodology known from the art. A non-limiting list of protecting groups include benzyl; substituted benzyl; alkylcarbonyls and alkoxycarbonyls (e.g., t-butoxycarbonyl (BOC), acetyl, or isobutyryl); arylalkylcarbonyls and arylalkoxycarbonyls (e.g., benzyloxycarbonyl); substituted methyl ether (e.g. methoxymethyl ether); substituted ethyl ether; a substituted benzyl ether; tetrahydropyranyl ether; silyls (e.g., trimethylsilyl, triethylsilyl, triisopropylsilyl, t-butyldimethylsilyl, tri-iso-propylsilyloxymethyl, [2-(trimethylsilyl)ethoxy]methyl or t-butyldiphenylsilyl); esters (e.g. benzoate ester); carbonates (e.g. methoxymethylcarbonate); sulfonates (e.g. tosylate or mesylate); acyclic ketal (e.g. dimethyl acetal); cyclic ketals (e.g., 1,3-dioxane, 1,3-dioxolanes, and those described herein); acyclic acetal; cyclic acetal (e.g., those described herein); acyclic hemiacetal; cyclic hemiacetal; cyclic dithioketals (e.g., 1,3-dithiane or 1,3-dithiolane); orthoesters (e.g., those described herein) and triarylmethyl groups (e.g., trityl; monomethoxytrityl (MMTr); 4,4′-dimethoxytrityl (DMTr); 4,4′,4″-trimethoxytrityl (TMTr); and those described herein).

The term “pharmaceutically acceptable salt” refers to a salt of a compound that does not cause significant irritation to an organism to which it is administered and does not abrogate the biological activity and properties of the compound. In some implementations, the salt is an acid addition salt of the compound. Pharmaceutical salts can be obtained by reacting a compound with inorganic acids such as hydrohalic acid (e.g., hydrochloric acid or hydrobromic acid), sulfuric acid, nitric acid and phosphoric acid. Pharmaceutical salts can also be obtained by reacting a compound with an organic acid such as aliphatic or aromatic carboxylic or sulfonic acids, for example formic, acetic, succinic, lactic, malic, tartaric, citric, ascorbic, nicotinic, methanesulfonic, ethanesulfonic, p-toluensulfonic, salicylic or naphthalenesulfonic acid. Pharmaceutical salts can also be obtained by reacting a compound with a base to form a salt such as an ammonium salt, an alkali metal salt, such as a sodium or a potassium salt, an alkaline earth metal salt, such as a calcium or a magnesium salt, a salt of organic bases such as dicyclohexylamine, N-methyl-D-glucamine, tris(hydroxymethyl)methylamine, C₁-C₇ alkylamine, cyclohexylamine, triethanolamine, ethylenediamine, and salts with amino acids such as arginine and lysine.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity. The indefinite article “a” or “an” does not exclude a plurality. A single processor or other unit may fulfill the functions of several items recited in the claims. The mere fact that certain measures are recited in mutually different dependent claims does not indicate that a combination of these measures cannot be used to advantage. Any reference signs in the claims should not be construed as limiting the scope.

It is understood that, in any compound described herein having one or more chiral centers, if an absolute stereochemistry is not expressly indicated, then each center may independently be of R-configuration or S-configuration or a mixture thereof. Thus, the compounds provided herein may be enantiomerically pure, enantiomerically enriched, racemic mixture, diastereomerically pure, diastereomerically enriched, or a stereoisomeric mixture. In addition it is understood that, in any compound described herein having one or more double bond(s) generating geometrical isomers that can be defined as E or Z, each double bond may independently be E or Z a mixture thereof.

It is understood that the compounds described herein can be labeled isotopically. Substitution with isotopes such as deuterium may afford certain therapeutic advantages resulting from greater metabolic stability, such as, for example, increased in vivo half-life or reduced dosage requirements. Each chemical element as represented in a compound structure may include any isotope of said element. For example, in a compound structure a hydrogen atom may be explicitly disclosed or understood to be present in the compound. At any position of the compound that a hydrogen atom may be present, the hydrogen atom can be any isotope of hydrogen, including but not limited to hydrogen-1 (protium) and hydrogen-2 (deuterium). Thus, reference herein to a compound encompasses all potential isotopic forms unless the context clearly dictates otherwise.

It is understood that the methods and combinations described herein include crystalline forms (also known as polymorphs, which include the different crystal packing arrangements of the same elemental composition of a compound), amorphous phases, salts, solvates, and hydrates. In some implementations, the compounds described herein exist in solvated forms with pharmaceutically acceptable solvents such as water, ethanol, or the like. In other implementations, the compounds described herein exist in unsolvated form. Solvates contain either stoichiometric or non-stoichiometric amounts of a solvent, and may be formed during the process of crystallization with pharmaceutically acceptable solvents such as water, ethanol, or the like. Hydrates are formed when the solvent is water, or alcoholates are formed when the solvent is alcohol. In addition, the compounds provided herein can exist in unsolvated as well as solvated forms. In general, the solvated forms are considered equivalent to the unsolvated forms for the purposes of the compounds and methods provided herein.

### Methods of Preparation

The compounds disclosed herein may be synthesized by methods described below, or by modification of these methods. Some compounds of can be obtained commercially and/or prepared utilizing known synthetic procedures. General synthetic routes to the compounds, and some examples of starting materials used to synthesize the compounds of Formula (I), Formula (II), Formula (III) and Formula (IV) are shown and described herein. The routes shown and described herein are illustrative only and are not intended, nor are they to be construed, to limit the scope of the claims in any manner whatsoever. Those skilled in the art will be able to recognize modifications of the disclosed syntheses and to devise alternate routes based on the disclosures herein; all such modifications and alternate routes are within the scope of the claims.

### Synthesis of Compounds

The following example schemes are provided for the guidance of the reader, and collectively represent an example method for making the compounds encompassed herein. Furthermore, other methods for preparing compounds described herein will be readily apparent to the person of ordinary skill in the art in light of the following reaction schemes and examples. Unless otherwise indicated, all variables are as defined above.

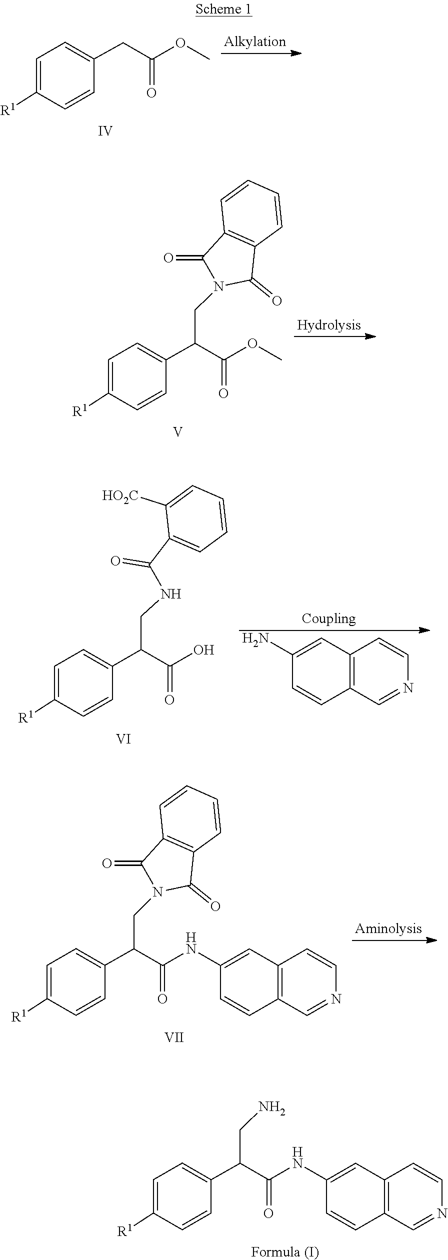

Compounds of Formula (I) can be prepared by following a sequence of steps as described in Bioorg. Med. Chem. Lett., 2016, 26, 2475-2480, which is incorporated herein by reference in its entirety. This sequence can start by carrying out an alkylation of compounds of Formula (IV) with N-(bromomethyl)phthalimide to afford compounds of Formula (V). Subsequent hydrolysis affords compounds of Formula (VII) which can then be subjected to aminolysis to afford compounds of Formula (I).

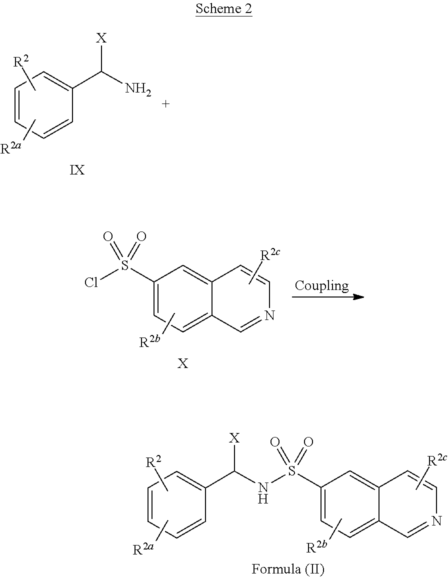

Compounds of Formula (II) can be prepared as described in U.S. Pat. No. 8,883,792, which is incorporated herein by reference in its entirety. This process involves coupling a primary amine of Formula (IX) with a sulfonyl chloride of Formula (X) to afford a sulfonamide of Formula (II).

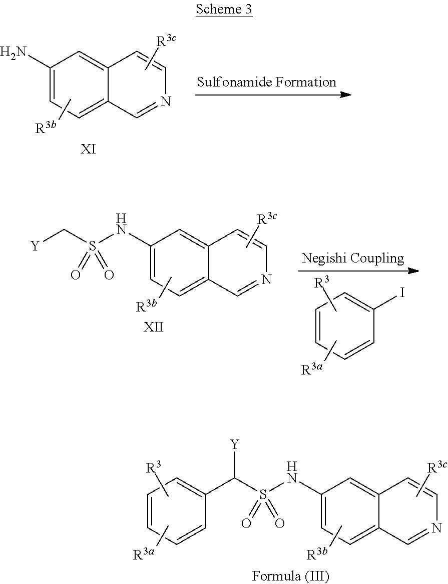

Compounds of Formula (III) can be prepared be starting with a compound of Formula (XI) and converting the primary amine into a sulfonamide of Formula (XII). Compounds of Formula (XII) can subsequently be subjected to Negishi coupling conditions, as described in Org. Lett., 2008, 10, 2817-2820, which is incorporated herein by reference in its entirety. This coupling affords compounds of Formula (III).

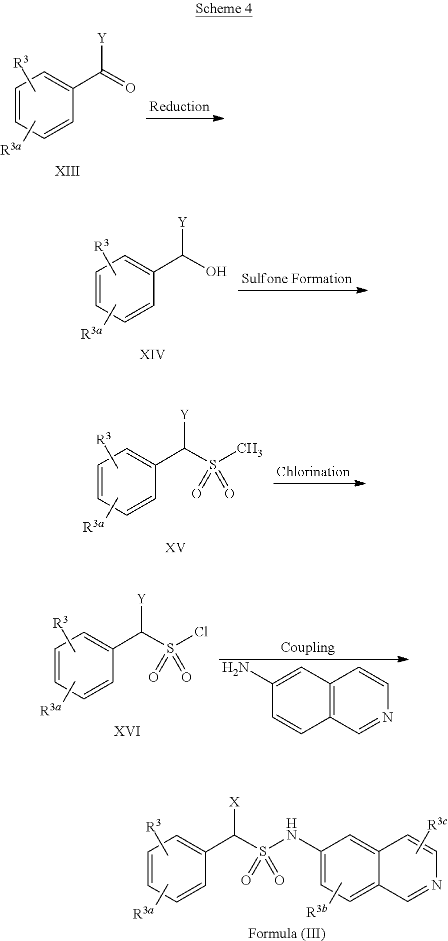

In an alternative sequence, certain compounds of Formula (III) are prepared by subjecting Compounds of Formula (XIII) to reduction conditions to afford compounds of Formula (XIV). Subsequent sulfone formation affords compounds of Formula (XV), which are then chlorinated for afford compounds of Formula (XVI). Then the compounds of Formula (XVI) are coupled with 6-aminoisoquinoline to afford compounds of Formula (III).

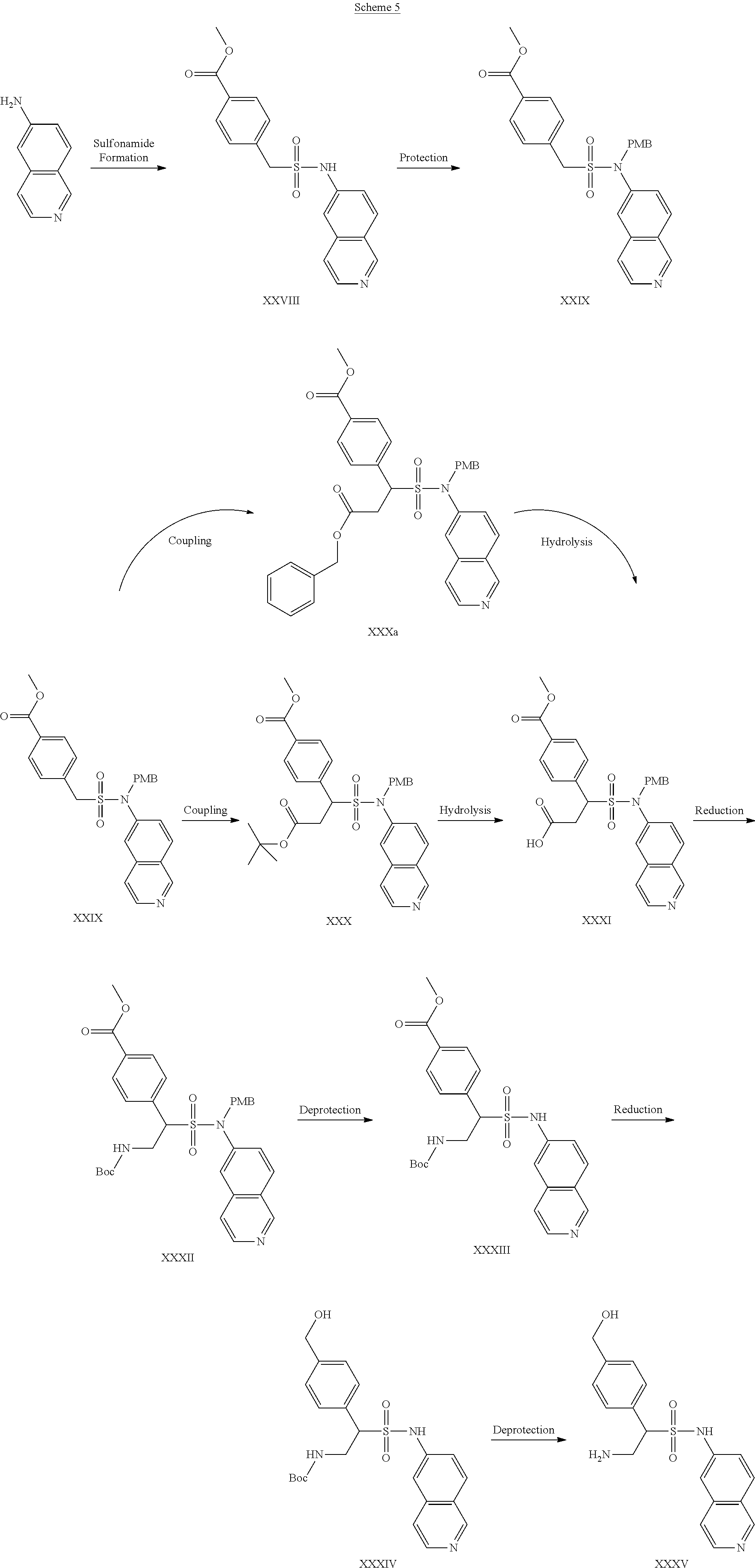

The synthesis of compound XXXV is an example of synthesis of compounds of Formula (III). Compound XXXV was prepared by starting with the conversion of 6-aminoisoquinoline to sulfonamide XXVIII. Compound XXVIII was then protected to form compound XXIX and coupled with either of two groups to form compound XXX or compound XXXa, before undergoing hydrolysis to form compound XXXI. The carboxylic acid was reduced to form compound XXXII, and the sulfonamide deprotected to provide compound XXXIII. Compound XXXIII was subsequently reduced to form compound XXXIV, which was deprotected to afford compound XXXV.

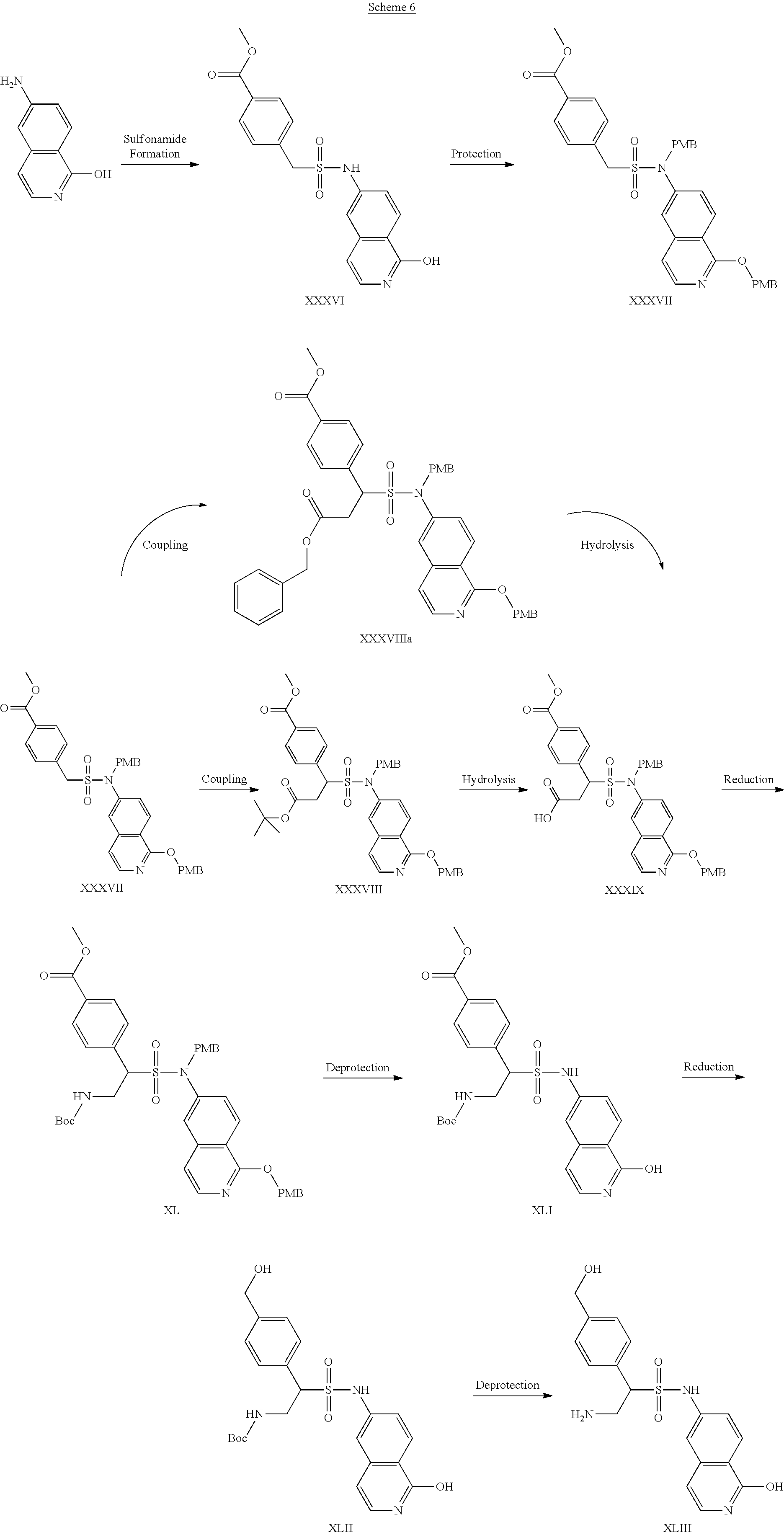

The synthesis of compound XLIII is another example of synthesis of compounds of Formula (III). Compound XLIII was prepared by starting with the conversion of 6-aminoisoquinolin-1-ol to sulfonamide XXXVI. Compound XXXVI was then protected to form compound XXXVII and coupled with either of two groups to form compound XXXVIII or compound XXXVIIIa, before undergoing hydrolysis to form compound XXXIX. The carboxylic acid was reduced to form compound XL, and the sulfonamide deprotected to provide compound XLI. Compound XLI was subsequently reduced to form compound XLII, which was deprotected to afford compound XLIII.

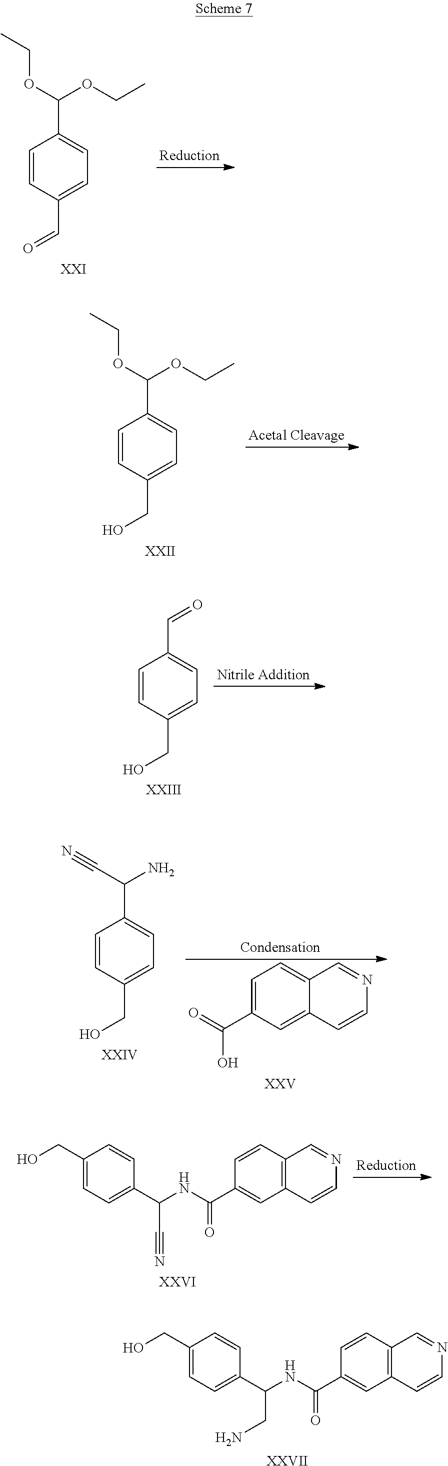

The synthesis of compound XXVII is an example of synthesis of compounds of Formula (IV). Compound XXVII can be prepared by starting with compound XXI and converting the aldehyde to a primary alcohol, as in compound XXII. Compound XXII is cleaved of an acetal to form aldehyde XXIII. The resulting aldehyde is subjected to nucleophilic addition of a nitrile through a Schiff base and subsequent reduction to a primary amine to form compound XXIV. Coupling of the successive amine and a carboxylic acid (compound XXV) forms a condensed product, compound XXVI. Final reduction of the nitrile to a primary amine affords compound XXVII.

### Administration and Pharmaceutical Compositions

Administration of the compounds disclosed herein or the pharmaceutically acceptable salts thereof can be via any of the accepted modes of administration for agents that serve similar utilities including, but not limited to, ocularly (including topically and intraocularly), orally, subcutaneously, intravenously, intranasally, topically, transdermally, intraperitoneally, intramuscularly, intrapulmonarilly, vaginally, or rectally.

The compounds useful as described above can be formulated into pharmaceutical compositions for use in treatment of these conditions. Standard pharmaceutical formulation techniques are used, such as those disclosed in Remington's The Science and Practice of Pharmacy, 21st Ed., Lippincott Williams & Wilkins (2005), incorporated by reference in its entirety. Accordingly, some implementations include pharmaceutical compositions comprising: (a) a safe and therapeutically effective amount of a compound described herein (including enantiomers, diastereoisomers, tautomers, polymorphs, and solvates thereof), or pharmaceutically acceptable salts thereof; and (b) a pharmaceutically acceptable carrier, diluent, excipient or combination thereof.

In addition to the selected compound useful as described above, some implementations include compositions containing a pharmaceutically-acceptable carrier. The term “pharmaceutically acceptable carrier” or “pharmaceutically acceptable excipient” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the active ingredient, its use in the therapeutic compositions is contemplated. In addition, various adjuvants such as are commonly used in the art may be included. Considerations for the inclusion of various components in pharmaceutical compositions are described, e.g., in Gilman et al. (Eds.) (1990); Goodman and Gilman's: The Pharmacological Basis of Therapeutics, 8th Ed., Pergamon Press, which is incorporated herein by reference in its entirety.

Some examples of substances, which can serve as pharmaceutically-acceptable carriers or components thereof, are sugars, such as lactose, glucose and sucrose; starches, such as corn starch and potato starch; cellulose and its derivatives, such as sodium carboxymethyl cellulose, ethyl cellulose, and methyl cellulose; powdered tragacanth; malt; gelatin; talc; solid lubricants, such as stearic acid and magnesium stearate; calcium sulfate; pharmaceutically acceptable oils including vegetable oils, such as peanut oil, cottonseed oil, sesame oil, olive oil, corn oil and oil of theobroma; polyols such as propylene glycol, glycerine, sorbitol, mannitol, and polyethylene glycol; alginic acid; emulsifiers, such as the TWEENS; wetting agents, such sodium lauryl sulfate; coloring agents; flavoring agents; tableting agents, stabilizers; antioxidants; preservatives; solvents including pyrogen-free water; isotonic saline; and phosphate buffer solutions.

The compositions as described above may be useful in any of a variety of suitable forms for a variety of routes for administration, for example, for ocular (including topical and intraocular), oral, nasal, rectal, topical (including transdermal), intracerebral, intracranial, intrathecal, intra-arterial, intravenous, intramuscular, or other parental routes of administration. The skilled artisan will appreciate that oral and nasal compositions comprise compositions that are administered by inhalation, and made using available methodologies. Depending upon the particular route of administration desired, a variety of pharmaceutically-acceptable carriers well-known in the art may be used. Pharmaceutically-acceptable carriers include, for example, solid or liquid fillers, diluents, hydrotropies, surface-active agents, and encapsulating substances. Optional pharmaceutically-active materials may be included, which do not substantially interfere with the inhibitory activity of the compound. The amount of carrier employed in conjunction with the compound is sufficient to provide a practical quantity of material for administration per unit dose of the compound. Techniques and compositions for making dosage forms useful in the methods described herein are described in the following references, all incorporated by reference herein: Modern Pharmaceutics, 4th Ed., Chapters 9 and 10 (Banker & Rhodes, editors, 2002); Lieberman et al., Pharmaceutical Dosage Forms: Tablets (1989); and Ansel, Introduction to Pharmaceutical Dosage Forms 8th Edition (2004).

A liquid composition, which is formulated for topical ocular or ophthalmic use, is formulated such that it can be administered topically to the eye. The comfort should be maximized as much as possible, although sometimes formulation considerations (e.g. drug stability) may necessitate less than optimal comfort. In the case that comfort cannot be maximized, the liquid should be formulated such that the liquid is tolerable to the patient for topical ophthalmic use. Additionally, an ophthalmically acceptable liquid should either be packaged for single use, or contain a preservative to prevent contamination over multiple uses.

For ophthalmic application, solutions or medicaments are often prepared using a physiological saline solution as a major vehicle. Ophthalmic solutions should preferably be maintained at a comfortable pH with an appropriate buffer system. The formulations may also contain conventional, pharmaceutically acceptable preservatives, stabilizers and surfactants. Ophthalmic solutions for administration as drops to the outer eye or cornea may include the drug at a concentration of about 0.001% to about 0.5%, including about about 0.01% to about 0.3%, about 0.01% to about 0.05%, and about 0.005% to about 0.01%.

Ophthalmic administration may also be accomplished with the use of an ocular implant. The implant may reside within the eye and be configured to deliver one or more drugs to anterior chamber of the eye in a controlled fashion or the implant may reside within and be configured to deliver one or more drugs to the posterior region of the eye in a controlled fashion. The implant may be configured to simultaneously deliver drugs to both the anterior and posterior region of the eye in a controlled fashion. The implant may be a punctal implant residing at least partially in a nasolacrimal duct. This advantageously allows release of the therapeutic agent from the implant, but prevents the drainage of tears from washing the drug down the nasolacrimal duct. The drug in the implant may be in solid or liquid form, including oils or solutions.

Various oral dosage forms can be used, including such solid forms as tablets, capsules, granules and bulk powders. Tablets can be compressed, tablet triturates, enteric-coated, sugar-coated, film-coated, or multiple-compressed, containing suitable binders, lubricants, diluents, disintegrating agents, coloring agents, flavoring agents, flow-inducing agents, and melting agents. Liquid oral dosage forms include aqueous solutions, emulsions, suspensions, solutions and/or suspensions reconstituted from non-effervescent granules, and effervescent preparations reconstituted from effervescent granules, containing suitable solvents, preservatives, emulsifying agents, suspending agents, diluents, sweeteners, melting agents, coloring agents and flavoring agents.

The pharmaceutically-acceptable carrier suitable for the preparation of unit dosage forms for peroral administration is well-known in the art. Tablets typically comprise conventional pharmaceutically-compatible adjuvants as inert diluents, such as calcium carbonate, sodium carbonate, mannitol, lactose and cellulose; binders such as starch, gelatin and sucrose; disintegrants such as starch, alginic acid and croscarmelose; lubricants such as magnesium stearate, stearic acid and talc. Glidants such as silicon dioxide can be used to improve flow characteristics of the powder mixture. Coloring agents, such as the FD&C dyes, can be added for appearance. Sweeteners and flavoring agents, such as aspartame, saccharin, menthol, peppermint, and fruit flavors, are useful adjuvants for chewable tablets. Capsules typically comprise one or more solid diluents disclosed above. The selection of carrier components depends on secondary considerations like taste, cost, and shelf stability, which are not critical, and can be readily made by a person skilled in the art.

Peroral compositions also include liquid solutions, emulsions, suspensions, and the like. The pharmaceutically-acceptable carriers suitable for preparation of such compositions are well known in the art. Typical components of carriers for syrups, elixirs, emulsions and suspensions include ethanol, glycerol, propylene glycol, polyethylene glycol, liquid sucrose, sorbitol and water. For a suspension, typical suspending agents include methyl cellulose, sodium carboxymethyl cellulose, AVICEL RC-591, tragacanth and sodium alginate; typical wetting agents include lecithin and polysorbate 80; and typical preservatives include methyl paraben and sodium benzoate. Peroral liquid compositions may also contain one or more components such as sweeteners, flavoring agents and colorants disclosed above.

Such compositions may also be coated by conventional methods, typically with pH or time-dependent coatings, such that the subject compound is released in the gastrointestinal tract in the vicinity of the desired topical application, or at various times to extend the desired action. Such dosage forms typically include, but are not limited to, one or more of cellulose acetate phthalate, polyvinylacetate phthalate, hydroxypropyl methyl cellulose phthalate, ethyl cellulose, Eudragit coatings, waxes and shellac.

Other compositions useful for attaining systemic delivery of the subject compounds include sublingual, buccal and nasal dosage forms. Such compositions typically comprise one or more of soluble filler substances such as sucrose, sorbitol and mannitol; and binders such as acacia, microcrystalline cellulose, carboxymethyl cellulose and hydroxypropyl methyl cellulose. Glidants, lubricants, sweeteners, colorants, antioxidants and flavoring agents disclosed above may also be included.

Preservatives that may be used in the pharmaceutical compositions disclosed herein include, but are not limited to, benzalkonium chloride, PHMB, chlorobutanol, thimerosal, phenylmercuric, acetate and phenylmercuric nitrate. A useful surfactant is, for example, Tween 80. Likewise, various useful vehicles may be used in the ophthalmic preparations disclosed herein. These vehicles include, but are not limited to, polyvinyl alcohol, povidone, hydroxypropyl methyl cellulose, poloxamers, carboxymethyl cellulose, hydroxyethyl cellulose and purified water.

Tonicity adjustors may be added as needed or convenient. They include, but are not limited to, salts, particularly sodium chloride, potassium chloride, mannitol and glycerin, or any other suitable ophthalmically acceptable tonicity adjustor.

Various buffers and means for adjusting pH may be used so long as the resulting preparation is ophthalmically acceptable. For many compositions, the pH will be between 4 and 9. Accordingly, buffers include acetate buffers, citrate buffers, phosphate buffers and borate buffers. Acids or bases may be used to adjust the pH of these formulations as needed.

In a similar vein, an ophthalmically acceptable antioxidant includes, but is not limited to, sodium metabisulfite, sodium thiosulfate, acetylcysteine, butylated hydroxyanisole and butylated hydroxytoluene.

Other excipient components, which may be included in the ophthalmic preparations, are chelating agents. A useful chelating agent is edetate disodium, although other chelating agents may also be used in place or in conjunction with it.

For topical use, creams, ointments, gels, solutions or suspensions, etc., containing the compound disclosed herein are employed. Topical formulations may generally be comprised of a pharmaceutical carrier, co-solvent, emulsifier, penetration enhancer, preservative system, and emollient.

For intravenous administration, the compounds and compositions described herein may be dissolved or dispersed in a pharmaceutically acceptable diluent, such as a saline or dextrose solution. Suitable excipients may be included to achieve the desired pH, including but not limited to NaOH, sodium carbonate, sodium acetate, HCl, and citric acid. In various implementations, the pH of the final composition ranges from 2 to 8, or preferably from 4 to 7. Antioxidant excipients may include sodium bisulfite, acetone sodium bisulfite, sodium formaldehyde, sulfoxylate, thiourea, and EDTA. Other non-limiting examples of suitable excipients found in the final intravenous composition may include sodium or potassium phosphates, citric acid, tartaric acid, gelatin, and carbohydrates such as dextrose, mannitol, and dextran. Further acceptable excipients are described in Powell, et al., Compendium of Excipients for Parenteral Formulations, PDA J Pharm Sci and Tech 1998, 52 238-311 and Nema et al., Excipients and Their Role in Approved Injectable Products: Current Usage and Future Directions, PDA J Pharm Sci and Tech 2011, 65 287-332, both of which are incorporated herein by reference in their entirety. Antimicrobial agents may also be included to achieve a bacteriostatic or fungistatic solution, including but not limited to phenylmercuric nitrate, thimerosal, benzethonium chloride, benzalkonium chloride, phenol, cresol, and chlorobutanol.

The compositions for intravenous administration may be provided to caregivers in the form of one more solids that are reconstituted with a suitable diluent such as sterile water, saline or dextrose in water shortly prior to administration. In other implementations, the compositions are provided in solution ready to administer parenterally. In still other implementations, the compositions are provided in a solution that is further diluted prior to administration. In implementations that include administering a combination of a compound described herein and another agent, the combination may be provided to caregivers as a mixture, or the caregivers may mix the two agents prior to administration, or the two agents may be administered separately.

The compounds are administered at a therapeutically effective dosage. The actual dose of the active compounds described herein depends on the specific compound, and on the condition to be treated; the selection of the appropriate dose is well within the knowledge of the skilled artisan. While human dosage levels have yet to be optimized for the compounds described herein, if the compound is in the form of a solution for topical ophthalmic use a daily dose may be one to three drops of a solution of the drug once or twice daily. If the compound is in an ophthalmic implant, the implant may be adapted to deliver drug at a rate of about 0.05 μg/day to about 5 μg/day, about 0.05 μg/day to about 3 μg/day, or about 0.05 μg/day to about 2 μg/day is achieved. In other implementations, an elution rate of about 2 μg/day to about 5 μg/day, about 4 μg/day to about 7 μg/day, or about 6 μg/day to about 10 μg/day is achieved. In other implementations, an elution rate of about 1 μg/day to about 4 μg/day, about 3 μg/day to about 6 μg/day, or about 7 μg/day to about 10 μg/day is achieved. In still other implementations, an elution rate of about 0.05 μg/day to about 1 μg/day, including 0.06, 0.07, 0.08, 0.09, 0.1, 0.2, 0.3, 0.4, 0.5, 0.6, 0.7, 0.8, or 0.9 μg/day is achieved. If the compound is delivered systemically, a daily dose may be from about 0.25 mg/kg to about 120 mg/kg or more of body weight, from about 0.5 mg/kg or less to about 70 mg/kg, from about 1.0 mg/kg to about 50 mg/kg of body weight, or from about 1.5 mg/kg to about 10 mg/kg of body weight. Thus, for administration to a 70 kg person, the dosage range would be from about 17 mg per day to about 8000 mg per day, from about 35 mg per day or less to about 7000 mg per day or more, from about 70 mg per day to about 6000 mg per day, from about 100 mg per day to about 5000 mg per day, or from about 200 mg to about 3000 mg per day. The amount of active compound administered will, of course, be dependent on the subject and disease state being treated, the severity of the affliction, the manner and schedule of administration and the judgment of the prescribing physician.

### ADDITIONAL EXAMPLES

### General Procedures

Materials used in the preparation of the compounds described herein may be made my known methods or commercially available. It will be apparent to the skilled artisan that methods for preparing precursors and functionality related to the compounds claimed herein are generally described in the literature. In these reactions, it is also possible to make use of variants which are themselves known to those of ordinary skill in this art, but are not mentioned in greater detail. The skilled artisan given the literature and this disclosure is well equipped to prepare any of the compounds.

It is recognized that the skilled artisan in the art of organic chemistry can readily carry out manipulations without further direction, that is, it is well within the scope and practice of the skilled artisan to carry out these manipulations. These include reduction of carbonyl compounds to their corresponding alcohols, oxidations, acylations, aromatic substitutions, both electrophilic and nucleophilic, etherifications, esterification and saponification and the like. These manipulations are discussed in standard texts such as March Advanced Organic Chemistry (Wiley), Carey and Sundberg, Advanced Organic Chemistry (incorporated herein by reference in their entirety) and the like.

The skilled artisan will readily appreciate that certain reactions are best carried out when other functionality is masked or protected in the molecule, thus avoiding any undesirable side reactions and/or increasing the yield of the reaction. Often the skilled artisan utilizes protecting groups to accomplish such increased yields or to avoid the undesired reactions. These reactions are found in the literature and are also well within the scope of the skilled artisan. Examples of many of these manipulations can be found for example in T. Greene and P. Wuts Protecting Groups in Organic Synthesis, 4th Ed., John Wiley & Sons (2007), incorporated herein by reference in its entirety.

The following example schemes are provided for the guidance of the reader, and represent preferred methods for making the compounds exemplified herein. These methods are not limiting, and it will be apparent that other routes may be employed to prepare these compounds. Such methods specifically include solid phase based chemistries, including combinatorial chemistry. The skilled artisan is thoroughly equipped to prepare these compounds by those methods given the literature and this disclosure. The compound numberings used in the synthetic schemes depicted below are meant for those specific schemes only, and should not be construed as or confused with same numberings in other sections of the application.

Trademarks used herein are examples only and reflect illustrative materials used at the time of the invention. The skilled artisan will recognize that variations in lot, manufacturing processes, and the like, are expected. Hence the examples, and the trademarks used in them are non-limiting, and they are not intended to be limiting, but are merely an illustration of how a skilled artisan may choose to perform one or more of the implementations of the invention.

The following abbreviations have the indicated meanings:
DCM=dichloromethane
DEAD=diethyl axodicarboxylate
DMAP=4-(dimethylamino)pyridine
EtOH=ethanol
EDAC=N-(3-dimethylaminopropyl)-N′-ethylcarbodiimide
LiHDMS=lithim bis(trimethylsilyl)amide
MeOH=methanol
Pd(OAc)₂=palladium(II) acetate
TBAB=tetrabutylammonium bromide
TBAF=tetrabutylammonium fluoride
TEA=triethylamine
THF=tetrahydrofuran
X-phos=2-dicyclohexylphosphino-2′, 4′, 6′-triisopropylbiphenyl

The following example schemes are provided for the guidance of the reader, and collectively represent an example method for making the compounds provided herein. Furthermore, other methods for preparing compounds described herein will be readily apparent to the person of ordinary skill in the art in light of the following reaction schemes and examples. Unless otherwise indicated, all variables are as defined above.

### Example 1

### 4-(2-amino-1-(N-(isoquinolin-6-yl)sulfamoyl)ethyl)phenyl]methyl 2,4-dimethylbenzoate (1)

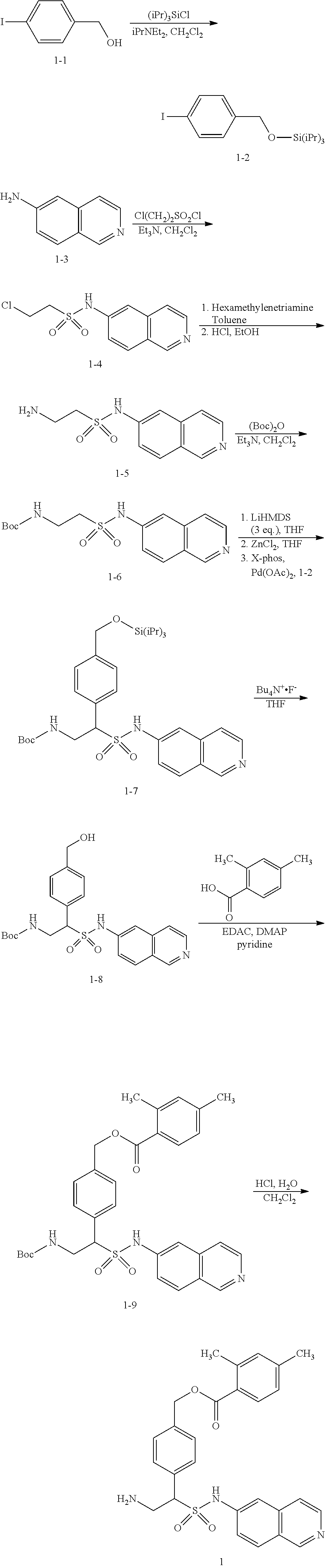

To a solution of 1-1 in DCM and iPrNEt₂, (iPr)₃SiCl is added. Workup of the resulting mixture affords 1-2.

To a solution of 1-3 in DCM and TEA, Cl(CH₂)₂SO₂Cl is added. Workup of the resulting mixture affords 1-4.

To a solution of 1-4 in toluene, hexamethylenetriamine is added and the mixture is allowed to stir. Then, HCl and EtOH are added to the mixture. Workup of the resulting mixture affords 1-5.

To a solution of 1-5 in DCM and TEA, (Boc)₂O is added. Workup of the resulting mixture affords 1-6.

To a solution of 1-6 in THF, 3.0 equivalents of LiHMDS is added and the mixture is allowed to stir. Then ZnCl₂ in THF is added and the mixture is allowed to stir. Then, X-phos, Pd(OAc)₂, and 1-2 are added to the mixture. Workup of the resulting mixture affords 1-7.

To a solution of 1-7 in THF, TBAF is added. Workup of the resulting mixture affords 1-8.

To a solution of 1-8 in pyridine, EDAC and DMAP are added. Workup of the resulting mixture affords 1-9.

To a solution of 1-9 in DCM, aqueous HCl is added. Workup of the resulting mixture affords 1.

### Example 2

### 4-(2-amino-1-(N-(isoquinolin-6-yl)sulfamoyl)ethyl) phenyl]methyl 2,4-dimethylbenzoate (1)

Compound 1 (described in Example 1) may also be prepared using the following synthetic sequence.

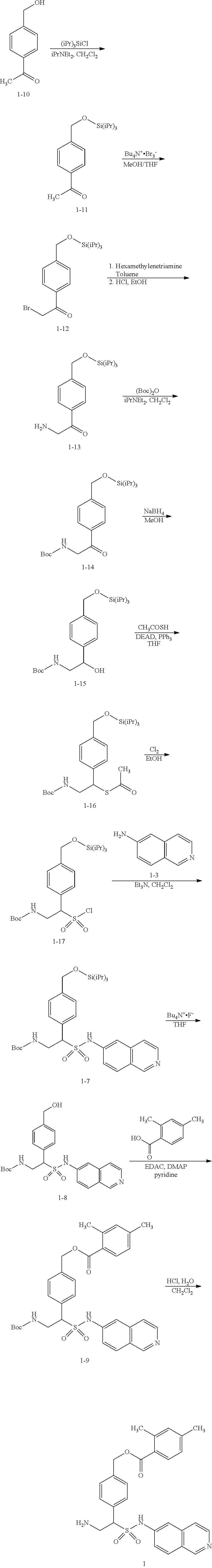

To a solution of 1-10 in DCM and iPrNEt₂, (iPr)₃SiCl is added. Workup of the resulting mixture affords 1-11.

To a solution of 1-11 in MeOH and THF, TBAB is added. Workup of the resulting mixture affords 1-12.

To a solution of 1-13 in DCM and iPrNEt₂, (Boc)₂O is added. Workup of the resulting mixture affords 1-14.

To a solution of 1-14 in MeOH, NaBH₄ is added. Workup of the resulting mixture affords 1-15.

To a solution of 1-15 in THF, CH₃COSH, DEAD, and PPH₃ are added.

Workup of the resulting mixture affords 1-16.

To a solution of 1-16 in EtOH, Cl₂ is added. Workup of the resulting mixture affords 1-17.

To a solution of 1-17 in DCM and TEA, 1-3 is added. Workup of the resulting mixture affords 1-7.

To a solution of 1-7 in THF, TBAF is added. Workup of the resulting mixture affords 1-8.

To a solution of 1-8 in pyridine, EDAC and DMAP are added. Workup of the resulting mixture affords 1-9.

To a solution of 1-9 in DCM, aqueous HCl is added. Workup of the resulting mixture affords 1.

It is to be understood that the term “embodiment” as used herein refers to an aspect or implementation of what is disclosed herein, and that embodiments may be combined with one another.

While certain implementations of the inventions have been described, these implementations have been presented by way of example only, and are not intended to limit the scope of the disclosure.

For purposes of this disclosure, certain aspects, advantages, and novel features are described herein. Not necessarily all such advantages may be achieved in accordance with any particular embodiment or implementation.

Conditional language, such as “can,” “could,” “might,” or “may,” unless specifically stated otherwise, or otherwise understood within the context as used, is generally intended to convey that certain implementations include, while other implementations do not include, certain features, functional groups, elements, and/or steps. Thus, such conditional language is not generally intended to imply that features, elements, and/or steps are in any way required for one or more embodiments or that one or more embodiments necessarily include logic for deciding, with or without user input or prompting, whether these features, elements, and/or steps are included or are to be performed in any particular embodiment.

Language of degree used herein, such as the terms “approximately,” “about,” “generally,” and “substantially” as used herein represent a value, amount, or characteristic close to the stated value, amount, or characteristic that still performs a desired function or achieves a desired result. For example, the terms “approximately”, “about”, “generally,” and “substantially” may refer to an amount that is within less than 10% of, within less than 5% of, within less than 1% of, within less than 0.1% of, and within less than 0.01% of the stated amount.

Unless otherwise defined, all terms (including technical and scientific terms) are to be given their ordinary and customary meaning to a person of ordinary skill in the art and are not to be limited to a special or customized meaning unless expressly so defined herein. It should be noted that the use of particular terminology when describing certain features or aspects of the disclosure should not be taken to imply that the terminology is being re-defined herein to be restricted to include any specific characteristics of the features or aspects of the disclosure with which that terminology is associated. Terms and phrases used in this application, and variations thereof, especially in the appended claims, unless otherwise expressly stated, should be construed as open ended as opposed to limiting. As examples of the foregoing, the term ‘including’ should be read to mean ‘including, without limitation,’ ‘including but not limited to,’ or the like; the term ‘comprising’ as used herein is synonymous with ‘including,’ ‘containing,’ or ‘characterized by,’ and is inclusive or open-ended and does not exclude additional, unrecited elements or method steps; “comprising” is intended to also include the narrower terms “consisting of” and “consisting essentially of,” the latter term meaning that the scope is limited to the recited elements or steps and any others that do not materially affect the basic and novel characteristics of what is already recited; the term ‘having’ should be interpreted as ‘having at least;’ the term ‘includes’ should be interpreted as ‘includes but is not limited to;’ the term ‘example’ is used to provide exemplary instances of the item in discussion, not an exhaustive or limiting list thereof; adjectives such as ‘known’, ‘normal’, ‘standard’, and terms of similar meaning should not be construed as limiting the item described to a given time period or to an item available as of a given time, but instead should be read to encompass known, normal, or standard technologies that may be available or known now or at any time in the future; and use of terms like ‘preferably,’ ‘preferred,’ ‘desired,’ or ‘desirable,’ and words of similar meaning should not be understood as implying that certain features are critical, essential, or even important to the structure or function of the invention, but instead as merely intended to highlight alternative or additional features that may or may not be utilized in particular implementations of the invention. Likewise, a group of items linked with the conjunction “and” should not be read as requiring that each and every one of those items be present in the grouping, but rather should be read as ‘and/or’ unless expressly stated otherwise. Similarly, a group of items linked with the conjunction ‘or’ should not be read as requiring mutual exclusivity among that group, but rather should be read as ‘and/or’ unless expressly stated otherwise.

Where a range of values is provided, it is to be understood that the upper and lower limit and each intervening value between the upper and lower limit of the range is disclosed.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity. The indefinite article “a” or “an” does not exclude a plurality. The mere fact that certain measures are recited in mutually different dependent claims does not indicate that a combination of these measures cannot be used to advantage.

It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations. In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations). Furthermore, in those instances where a convention analogous to “at least one of A, B, and C, etc.” is used, in general such a construction is intended in the sense one having skill in the art would understand the convention, e.g., as including any combination of the listed items, including single members (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

All references cited herein are incorporated herein by reference in their entirety. To the extent publications and patents or patent applications incorporated by reference contradict the disclosure contained in the specification, the specification is intended to supersede and/or take precedence over any such contradictory material.

The scope of the present disclosure is not intended to be limited by the specific disclosures of preferred implementations in this section or elsewhere in this specification, and may be defined by claims as presented in this section or elsewhere in this specification or as presented in the future. The language of the claims is to be interpreted broadly based on the language employed in the claims and not limited to the examples described in the present specification or during the prosecution of the application, which examples are to be construed as non-exclusive

1. A compound of Formula (III), or a pharmaceutically acceptable salt, solvate, or prodrug thereof, having the structure: 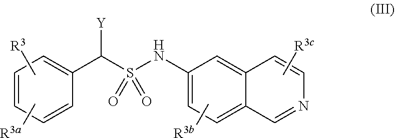
wherein:
Y is selected from the group consisting of hydrogen, an optionally substituted C₁-C₄ alkyl, an optionally substituted C₂-C₄ alkenyl, an optionally substituted C₂-C₄ alkynyl, cyano, halogen, hydroxy, an optionally substituted aryl, and an optionally substituted aminoalkyl;
R³ and R^{3a} are each independently selected from the group consisting of hydrogen, an optionally substituted C₁-C₄ alkyl, an optionally substituted C₂-C₄ alkenyl, an optionally substituted C₂-C₄ alkynyl, cyano, halogen, hydroxy, an optionally substituted aryl, 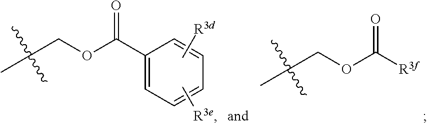
wherein R^{3d} and R^{3e} are each independently selected from the group consisting of hydrogen, an optionally substituted C₁-C₄ alkyl, an optionally substituted C₂-C₄ alkenyl, an optionally substituted C₂-C₄ alkynyl, cyano, halogen, hydroxy, and an optionally substituted aminoalkyl;
R^{3f} is selected from the group consisting of an optionally substituted C₁-C₄ alkyl, an optionally substituted C₂-C₄ alkenyl, an optionally substituted C₂-C₄ alkynyl, an optionally substituted aryl, and an optionally substituted aralkyl; and
R^{3b} and R^{3c} are each independently selected from the group consisting of hydrogen, an optionally substituted C₁-C₄ alkyl, an optionally substituted C₂-C₄ alkenyl, an optionally substituted C₂-C₄ alkynyl, cyano, halogen, hydroxy, and an optionally substituted aminoalkyl.

2. The compound of claim 1, wherein R^{3b} and R^{3c} are each independently selected from the group consisting of hydrogen, hydroxy, and —CH₂CH₂NH₂.

3. The compound of claim 1, wherein R^{3b} and R^{3c} are both hydrogen.

4. The compound of claim 1, wherein Y is an optionally substituted aminoalkyl.

5. The compound of claim 4 wherein Y is —CH₂NH₂.

6. The compound of claim 1, wherein R³ and R^{3a} are each independently selected from the group consisting of hydrogen, an optionally substituted C₁-C₄ alkyl, halogen, an optionally substituted aryl, and

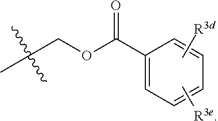

7. The compound of claim 6, wherein R³ is hydrogen and R^{3a} is selected from the group consisting of methyl, —CH₂OH, —Cl, naphthyl, 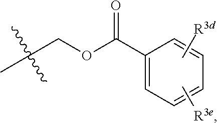
wherein R^{3d} and R^{3e} are each independently selected from the group consisting of hydrogen, methyl, and —CH₂CH₂NH₂.

8. The compound of claim 1, wherein R³ and R^{3a} are not both hydrogen.

9. The compound of claim 1, having the structure selected from the group consisting of:

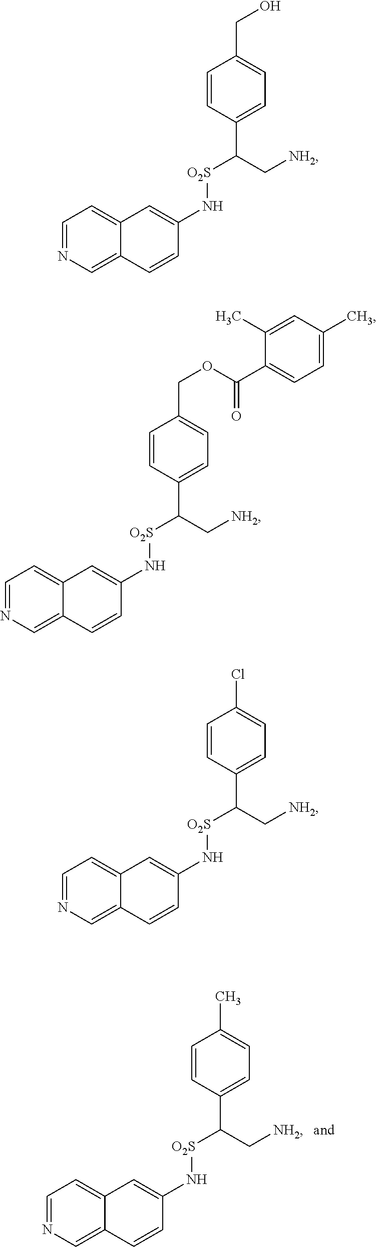 [SMILES: NCC(c1ccc(CO)cc1)S(=O)(=O)Nc1ccc2cnccc2c1 ; Cc1ccc(C(=O)OCc2ccc(C(CN)S(=O)(=O)Nc3ccc4cnccc4c3)cc2)c(C)c1 ; NCC(c1ccc(Cl)cc1)S(=O)(=O)Nc1ccc2cnccc2c1 ; Cc1ccc(C(CN)S(=O)(=O)Nc2ccc3cnccc3c2)cc1]

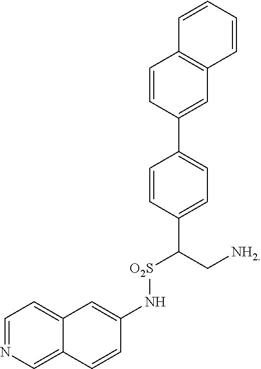 [SMILES: NCC(c1ccc(-c2ccc3ccccc3c2)cc1)S(=O)(=O)Nc1ccc2cnccc2c1]

10. The compound of claim 1 having the structure

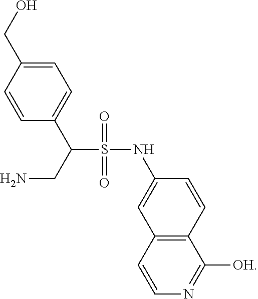 [SMILES: NCC(c1ccc(CO)cc1)S(=O)(=O)Nc1ccc2c(O)nccc2c1]

11. (canceled)

12. (canceled)

13. (canceled)

14. A compound of Formula (II), or a pharmaceutically acceptable salt, solvate, or prodrug thereof, having the structure: 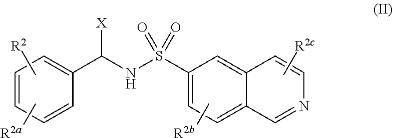
wherein:
X is selected from the group consisting of hydrogen, an optionally substituted C₁-C₄ alkyl, an optionally substituted C₂-C₄ alkenyl, an optionally substituted C₂-C₄ alkynyl, cyano, halogen, hydroxy, an optionally substituted aryl, and an optionally substituted aminoalkyl;
R² and R^{2a} are each independently selected from the group consisting of hydrogen, an optionally substituted C₁-C₄ alkyl, an optionally substituted C₂-C₄ alkenyl, an optionally substituted C₂-C₄ alkynyl, cyano, halogen, hydroxy, an optionally substituted aryl, 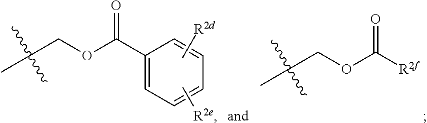
wherein R^{2d} and R^{2e} are each independently selected from the group consisting of hydrogen, an optionally substituted C₁-C₄ alkyl, an optionally substituted C₂-C₄ alkenyl, an optionally substituted C₂-C₄ alkynyl, cyano, halogen, hydroxy, and optionally substituted aminoalkyl;
R^{2f} is selected from the group consisting of an optionally substituted C₁-C₄ alkyl, an optionally substituted C₂-C₄ alkenyl, an optionally substituted C₂-C₄ alkynyl, an optionally substituted aryl, and an optionally substituted aralkyl; and
R^{2b} and R^{2c} are each independently selected from the group consisting of hydrogen, an optionally substituted C₁-C₄ alkyl, an optionally substituted C₂-C₄ alkenyl, an optionally substituted C₂-C₄ alkynyl, cyano, halogen, hydroxy, and an optionally substituted aminoalkyl.

15. The compound of claim 14, wherein R^{2b} and R^{2c} are each independently selected from the group consisting of hydrogen, hydroxy, and —CH₂CH₂NH₂.

16. The compound of claim 14, wherein R^{2b} and R^{2c} are both hydrogen.

17. The compound of claim 14, wherein X is an optionally substituted aminoalkyl.

18. The compound of claim 17, wherein X is —CH₂NH₂.

19. The compound of claim 14, wherein R² and R^{2a} are each independently selected from the group consisting of hydrogen, an optionally substituted C₁-C₄ alkyl, halogen, an optionally substituted aryl, and

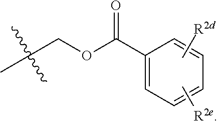

20. The compound of claim 19, wherein R² is hydrogen and R^{2a} is selected from the group consisting of methyl, —CH₂OH, —Cl, naphthyl, and 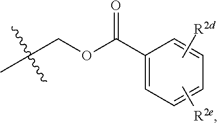
wherein R^{2d} and R^{2e} are each independently selected from the group consisting of hydrogen, methyl, and —CH₂CH₂NH₂.

21. The compound of claim 14, wherein R² and R^{2a} are not both hydrogen.

22. The compound of claim 14, having the structure selected from the group consisting of:

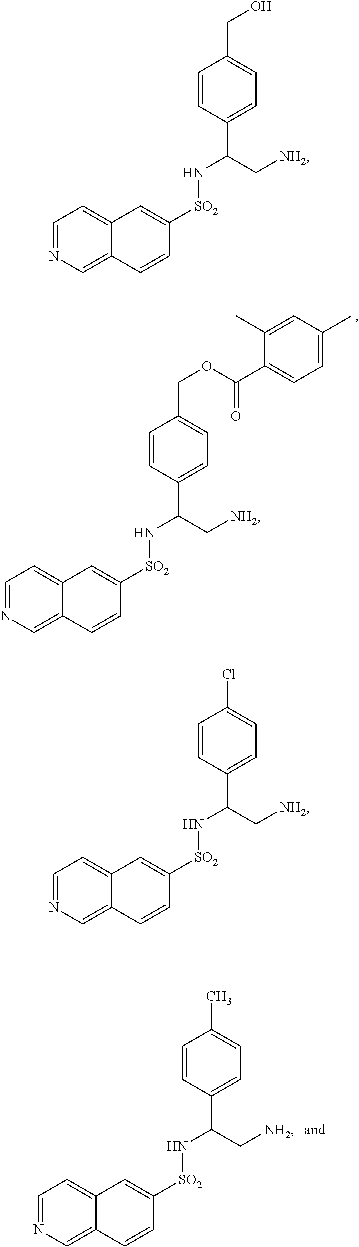 [SMILES: NCC(NS(=O)(=O)c1ccc2cnccc2c1)c1ccc(CO)cc1 ; Cc1ccc(C(=O)OCc2ccc(C(CN)NS(=O)(=O)c3ccc4cnccc4c3)cc2)c(C)c1 ; NCC(NS(=O)(=O)c1ccc2cnccc2c1)c1ccc(Cl)cc1 ; Cc1ccc(C(CN)NS(=O)(=O)c2ccc3cnccc3c2)cc1]

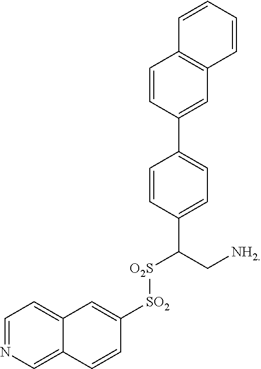 [SMILES: NCC(c1ccc(-c2ccc3ccccc3c2)cc1)S(=O)(=O)S(=O)(=O)c1ccc2cnccc2c1]

23. (canceled)

24. (canceled)

25. (canceled)

26. (canceled)

27. (canceled)

28. (canceled)

29. (canceled)

30. (canceled)

31. (canceled)

32. (canceled)
